(19) United States
(12) Patent Application Publication
(10) Pub. No.: US 2024/0216238 A1
(43) Pub. Date: Jul. 4, 2024

(54) SKIN AND HAIR CARE FORMULATIONS

(71) Applicant: Clarity Cosmetics Inc., Bethesda, MD (US)

(72) Inventors: Iris Rubin, Potomac, MD (US); John Anthony Garruto, Encinitas, CA (US); Bethany Ann McCarver, Oceanside, CA (US)

(21) Appl. No.: 18/230,100

(22) Filed: Aug. 3, 2023

Related U.S. Application Data
(60) Provisional application No. 63/370,413, filed on Aug. 4, 2022.

(51) Int. Cl. A61K 8/31; A61K 8/92; A61Q 5/00; A61Q 5/02; A61Q 19/10

(52) U.S. Cl. CPC A61K 8/31; A61K 8/922; A61K 8/925; A61Q 5/002; A61Q 5/02; A61Q 19/10; A61K 2800/30

(57) ABSTRACT

Provided for herein are embodiments of skin and/or hair cleansing and skin and/or hair treatment formulations. Several embodiments provide formulations for skin cleansing, including the skin of the scalp. In several embodiments, the formulations reduce hair loss and/or promote hair growth. In several embodiments, the formulations repair or heal damaged skin and/or hair. In several embodiments, the formulations improve the overall look, texture, strength and/or health of skin and/or hair and do so without the tendency to clog or block pores or irritate skin.

### CROSS-REFERENCE TO RELATED APPLICATIONS

This application claims priority to U.S. Provisional Patent Application No. 63/370,413, filed Aug. 4, 2022, the entire contents of which is incorporated by reference herein.

### BACKGROUND

Environmental exposure, aging, and residual components of hair and skin care products can have deleterious effects on a person's skin, including the skin of the scalp and other areas (e.g., face, neck, back etc.), such as redness, eczema, irritation, clogging of pores, hair loss and the like. There is a need for improved topical hair and skin care formulations that provide effective cleansing or treatment of hair and/or skin while also reducing deleterious effect for the user.

### SUMMARY

In several embodiments, cleansing formulations, serums, masks, and other cosmetic products to be used on skin (including skin of the scalp and/or non-scalp skin and/or hair) are provided. According to several embodiments, the topical formulations that are provided for herein show visible, beneficial dermatological results are provided.

In several embodiments, skin care formulations, include for example, liquid, serums, lotions, and/or creams are provided. Solid forms (e.g., jellies, powder, soaps) can also be provided, in several embodiments. Formulations provided for herein can act as one or more of a cleanser, moisturizer, sunscreen, anti-aging, anti-acne and/or skin brightener among other skin care benefits.

In several embodiments, there is provided a topical formulation, comprising water, sodium lauroyl methyl isethionate, acrylates copolymer, glycerin, cocamidopropylamine oxide, bisabolol, C13-C15 alkanes, and at least two of phenoxyethanol, ethylhexylglycerin, citric acid and aminomethyl propanol. In several embodiments, there is provided a topical formulation, comprising water, sodium lauroyl methyl isethionate, acrylates copolymer, glycerin, cocamidopropylamine oxide, bisabolol, C13-C15 alkanes, and at least two of a preservative, a skin conditioning agent, a pH modifier, and a buffer. In several embodiments, the water is present in an amount between about 60% and about 90% (e.g., about 60, 65, 70, 75, 80, 85, 90, or 95%, including amounts between those listed). In several embodiments, the sodium lauroyl methyl isethionate is present in an amount between about 3% and about 25% (e.g., about 3, 5, 7, 9, 10, 12, 15, 17, 20, 22, or 25%, including amounts between those listed). In several embodiments, the acrylates copolymer is present in an amount between about 0.1% and about 10% (e.g., about 0.1, 0.5, 1, 2, 3, 4, 5, 7, 8, 9, or 10%, including amounts between those listed). In several embodiments, the glycerin is present in an amount ranging between about 0.01 and about 10% (e.g., about 0.01, 0.05, 0.1, 0.5, 1, 2, 3, 4, 5, 7, 8, 9, or 10%, including amounts between those listed). In several embodiments, the cocamidopropylamine oxide is present in an amount between about 0.1% and about 10.0% (e.g., about 0.1, 0.5, 1, 2, 3, 4, 5, 7, 8, 9, or 10%, including amounts between those listed). In several embodiments, the bisabolol is present in an amount between about 0.01% and about 5% (e.g., about 0.01, 0.05, 0.1, 0.5, 1, 2, 3, 4, or 5%, including amounts between those listed). In several embodiments, the C13-C15 alkanes are present in an amount between 0.01% and 10% (e.g., about 0.01, 0.05, 0.1, 0.5, 1, 2, 3, 4, 5, 7, 8, 9, or 10%, including amounts between those listed).

Additionally provided for herein, in several embodiments, is a topical formulation, comprising water, wherein the water is present in an amount between about 60% and about 90% (e.g., about 60, 65, 70, 75, 80, 85, 90, or 95%, including amounts between those listed), sodium lauroyl methyl isethionate, wherein the sodium lauroyl methyl isethionate is present in an amount between about 3% and about 25% (e.g., about 3, 5, 7, 9, 10, 12, 15, 17, 20, 22, or 25%, including amounts between those listed), acrylates copolymer, wherein the acrylates copolymer is present in an amount between about 0.1% and about 10% (e.g., about 0.1, 0.5, 1, 2, 3, 4, 5, 7, 8, 9, or 10%, including amounts between those listed), glycerin, wherein the glycerin is present in an amount ranging between about 0.01 and about 10% (e.g., about 0.01, 0.05, 0.1, 0.5, 1, 2, 3, 4, 5, 7, 8, 9, or 10%, including amounts between those listed), and cocamidopropylamine oxide, wherein the cocamidopropylamine oxide is present in an amount between about 0.1% and about 10.0% (e.g., about 0.1, 0.5, 1, 2, 3, 4, 5, 7, 8, 9, or 10%, including amounts between those listed).

In several embodiments, the sodium lauroyl methyl isethionate is present in an amount ranging from about 8% to about 18% (e.g., about 8, 9, 10, 12, 14, 16, or 18%, including amounts between those listed). In several embodiments, wherein the phenoxyethanol and ethylhexylglycerin are present in a combined amount of between about 0.25% and about 5% (e.g., about 0.25, 0.5, 1, 2, 3, 4, or 5%, including amounts between those listed), and citric acid is present in an amount between about 0.01% and about 2.5% (e.g., about 0.01, 0.05, 0.1, 0.5, 1, 1.5, 2, or 2.5%, including amounts between those listed).

In several embodiments, the formulation further comprises guar hydroxypropyltrimonium chloride. In several embodiments, the guar hydroxypropyltrimonium chloride is present in an amount between 0.05-2.5% (e.g., about 0.05, 0.1, 0.5, 1, 1.5, 2, or 2.5%, including amounts between those listed).

In several embodiments, the formulation further comprises one or both of sodium hydroxide and sodium benzoate. In several embodiments, the formulation further comprises both of sodium hydroxide and sodium benzoate. In several embodiments, the formulation further comprises sodium hydroxide in an amount ranging between about 0.001% and about 0.5% (e.g., about 0.001, 0.0025, 0.005, 0.01, 0.025, 0.05, 0.1, 0.25, or 0.5%, including amounts between those listed). In several embodiments, the formulation further comprises sodium benzoate in an amount ranging between about 0.1% and 2.5% (e.g., about 0.1, 0.5, 1, 1.5, 2, or 2.5%, including amounts between those listed).

In several embodiments, the formulation further comprises at least one vitamin E compound. In several embodiments, the vitamin E compound comprises tocopherol, wherein the tocopherol is present in an amount ranging between about 0.0005% and about 1.0% (e.g., about 0.0005, 0.001, 0.005, 0.01, 0.05, 0.1, 0.5, or 1%, including amounts between those listed).

In several embodiments, the formulation further comprises at least one neutralizer of metal ions. In several embodiments, the neutralizer of metal ions functions via chelation of the ions. In several embodiments, the neutralizer comprises trisodium ethylenediamine disuccinate. In several embodiments, the neutralizer is present in an amount ranging from about 0.1% to about 1.0% (e.g., about 0.1, 0.5, 0.75 or 1%, including amounts between those listed).

In several embodiments, the formulation further comprises squalane and/or squalene. In several embodiments, the formulation comprises squalane in an amount ranging between about 0.1% and about 4.0% (e.g., about 0.1, 0.5, 1, 2, 3, or 4%, including amounts between those listed). In several embodiments, the formulation further comprises lauryl lactyl lactate. In several embodiments, the lauryl lactyl lactate is present in an amount ranging between about 0.1% and about 3% (e.g., about 0.1, 0.5, 1, 1.5, 2, 2.5, or 3%, including amounts between those listed). In several embodiments, the sodium lauroyl methyl isethionate is present in an amount ranging from about 5% to about 15% (e.g., about 5, 7, 8, 9, 10, 11, 12, 13, 14, or 15%, including amounts between those listed).

In several embodiments, the formulation further comprises polyquaternium-7. In several embodiments, the polyquaternium-7 is present in an amount ranging from about 0.01% to about 2.5% (e.g., about 0.01, 0.05, 0.1, 0.5, 1, 1.5, 2, or 2.5%, including amounts between those listed). In several embodiments, the formulation further comprises disodium EDTA, wherein the disodium EDTA is present in an amount ranging from about 0.01% and about 2% (e.g., about 0.01, 0.05, 0.1, 0.5, 0.6, 1.0, 1.5, or 2%, including amounts between those listed).

In several embodiments, the topical formulation is formulated as a face wash. In several embodiments, the topical formulation is formulated as a body wash. In several embodiments, the topical formulation is fragrance free. In several embodiments, the formulation is safe for eczema-prone skin. However, in several embodiments, the formulation further comprises fragrance.

In several embodiments, the topical formulations are used in a method of cleansing skin, the method comprising applying a formulation, or instructing the application of a formulation, according to embodiments provided for herein. In several embodiments, the skin is dry. In several embodiments, the skin is oily. In several embodiments, the skin has wrinkles. In several embodiments, the skin has inflammation. In several embodiments, the skin has blemishes. In several embodiments, the skin is sun damaged. In several embodiments, the skin is healthy.

Also provided for herein is a use of the formulations disclosed herein for the cleansing of skin.

In several embodiments, there is provided for herein a topical formulation, wherein the formulation comprises water, glycerin, C13-C15 alkanes, bisabolol, propanediol caprylate, phenoxyethanol, and ethylhexylglycerin. In several embodiments, there is provided for herein a topical formulation, wherein the formulation comprises water, glycerin, C13-C15 alkanes, bisabolol, propanediol caprylate, and at least two of a preservative, an emollient, a pH modifier, and a buffer.

In several embodiments, there is provided a topical formulation, wherein the formulation comprises water, glycerin, C13-C15 alkanes, bisabolol, propanediol caprylate, sarcosine, ethylhexyl olivate, squalane, and at least one of an amino acid, an emulsifier and/or thickening agent, and a preservative.

In several embodiments, the glycerin is present in an amount ranging between about 0.001 and about 15.0% (e.g., about 0.001, 0.005, 0.01, 0.05, 0.1, 0.5, 1, 2, 3, 4, 5, 7, 9, 11, 13, or 15%, including amounts between those listed). In several embodiments, the C13-C15 alkanes are present in an amount from about 0.001 to about 15.0% (e.g., about 0.001, 0.005, 0.01, 0.05, 0.1, 0.5, 1, 2, 3, 4, 5, 7, 9, 11, 13, or 15%, including amounts between those listed). In several embodiments, the bisabolol is present in an amount between about 0.001% and about 10% (e.g., about 0.001, 0.005, 0.01, 0.05, 0.1, 0.5, 1, 2, 3, 4, 5, 7, 8, 9, or 10%, including amounts between those listed). In several embodiments, the propanediol caprylate is present in an amount between about 0.01% and about 5% (e.g., about 0.01, 0.05, 0.1, 0.5, 1, 2, 3, 4, or 5%, including amounts between those listed). In several embodiments, the phenoxyethanol is present in an amount between about 0.01% and about 5% (e.g., about 0.01, 0.05, 0.1, 0.5, 1, 2, 3, 4, or 5%, including amounts between those listed). In several embodiments, the ethylhexylglycerin is present in an amount between about 0.01% and about 5% (e.g., about 0.01, 0.05, 0.1, 0.5, 1, 2, 3, 4, or 5%, including amounts between those listed). In several embodiments, the sarcosine is present in an amount between about 0.001% and about 15.0% (e.g., about 0.001, 0.005, 0.01, 0.05, 0.1, 0.5, 1, 2, 3, 4, 5, 7, 9, 11, 13, or 15%, including amounts between those listed). In several embodiments, the ethylhexyl olivate is present in an amount between about 0.001% and about 15.0% (e.g., about 0.001, 0.005, 0.01, 0.05, 0.1, 0.5, 1, 2, 3, 4, 5, 7, 9, 11, 13, or 15%, including amounts between those listed). In several embodiments, the squalane is present in amount between about 0.001% and about 15.0% (e.g., about 0.001, 0.005, 0.01, 0.05, 0.1, 0.5, 1, 2, 3, 4, 5, 7, 9, 11, 13, or 15%, including amounts between those listed).

In several embodiments, the glycerin is present in an amount ranging between about 0.0.001% and about 15.0% (e.g., about 0.001, 0.005, 0.01, 0.05, 0.1, 0.5, 1, 2, 3, 4, 5, 7, 9, 11, 13, or 15%, including amounts between those listed). In several embodiments, the C13-C15 alkanes are present in an amount from about 0.001 to about 15%. (e.g., about 0.001, 0.005, 0.01, 0.05, 0.1, 0.5, 1, 2, 3, 4, 5, 7, 9, 11, 13, or 15%, including amounts between those listed). In several embodiments, the bisabolol is present in an amount between 0.001% and 5% (e.g., about 0.001, 0.005, 0.01, 0.05, 0.1, 0.5, 1, 2, 3, 4, or 5%, including amounts between those listed).

In several embodiments, the propanediol caprylate is present in an amount between about 0.01% and about 5.0% (e.g., about 0.01, 0.05, 0.1, 0.5, 1, 2, 3, 4, or 5%, including amounts between those listed). In several embodiments, the sarcosine is present in an amount between about 0.001% and about 10.0% (e.g., about 0.001, 0.005, 0.01, 0.05, 0.1, 0.5, 1, 2, 3, 4, 5, 7, 9, or 10%, including amounts between those listed). In several embodiments, the ethylhexyl olivate is present in an amount between about 0.001% and about 10.0% (e.g., about 0.001, 0.005, 0.01, 0.05, 0.1, 0.5, 1, 2, 3, 4, 5, 6, 7, 8, 9, or 10%, including amounts between those listed). In several embodiments, the squalane is present in amount between about 0.001% and about 15.0% (e.g., about 0.001, 0.005, 0.01, 0.05, 0.1, 0.5, 1, 2, 3, 4, 5, 7, 9, 11, 13, or 15%, including amounts between those listed).

In several embodiments, there is provided a topical formulation comprising water, propanediol caprylate, wherein the propanediol caprylate is present in an amount between about 0.01% and 5%, (e.g., about 0.01, 0.05, 0.1, 0.5, 1, 2, 3, 4, or 5%, including amounts between those listed) sarcosine, wherein the sarcosine is present in an amount between about 0.001% and about 10.0% (e.g., about 0.001, 0.005, 0.01, 0.05, 0.1, 0.5, 1, 2, 3, 4, 5, 6, 7, 8, 9, or 10%, including amounts between those listed), ethylhexyl olivate, wherein the ethylhexyl olivate is present in an amount between about 0.005% and about 10.0% (e.g., about 0.005, 0.01, 0.05, 0.1, 0.5, 1, 2, 3, 4, 5, 6, 7, 8, 9, or 10%, including amounts between those listed), squalene, wherein the squalene is present in amount between about 0.001% and about 10.0% (e.g., about 0.001, 0.005, 0.01, 0.05, 0.1, 0.5, 1, 2, 3, 4, 5, 6, 7, 8, 9, or 10%, including amounts between those listed), arginine, wherein the arginine is present in an amount between about 0.005% and about 5.0% (e.g., about 0.001, 0.005, 0.01, 0.05, 0.1, 0.5, 1, 2, 3, 4, or 5%, including amounts between those listed); and acrylates/C10-30 alkyl acrylate crosspolymer, wherein the acrylates/C10-30 alkyl acrylate crosspolymer is present in an amount between about 0.005% and about 5% (e.g., about 0.005, 0.01, 0.05, 0.1, 0.5, 1, 2, 3, 4, or 5%, including amounts between those listed).

In several embodiments, there is provided a topical formulation, wherein the formulation comprises water, propanediol caprylate, sarcosine, ethylhexyl olivate, squalene, at least one amino acid functioning as a skin moisturizing and/or anti-aging agent, an emulsifier or thickening agent, and optionally a preservative. In several embodiments, the formulation comprises a preservative.

In several embodiments, the formulation is formulated as a dry scalp treating serum. In several embodiments, the formulation is formulated as a dry scalp treating shampoo or conditioner. In several embodiments, the formulation is formulated as a dry scalp treating oil. In several embodiments, the formulation is fragrance free. However, in several embodiments, the formulation further comprises fragrance. In several embodiments, the formulation is safe for eczema-prone skin. In several embodiments, the formulation is used to treat dandruff, eczema, psoriasis, dermatitis, scalp irritation, and/or scalp folliculitis. In several embodiments, the formulation is used to prevent or treat scalp dryness, wherein the formulation yield improved scalp hydration, reduces flaking and/or the appearance of flakes, reduces scalp irritation, supports a healthy scalp microbial ecosystem, and/or protects the scalp barrier. In several embodiments, the formulation is used to maintain a healthy scalp. In several embodiments, the formulation optimizes and resets the scalp of a user with balanced hydration. In several embodiments, the formulation is fast absorbing and non-greasy.

In several embodiments, there is also provided a method for treating or preventing dry scalp comprising applying a formulation, or instructing the application of a formulation, according to the embodiments disclosed herein. In several embodiments, the skin of the scalp also comprises hair. In several embodiments, there is provided for herein a use of formulation provided for herein for treating or preventing dry, flaky and/or itchy scalp.

In several embodiments, there is provided for herein a topical formulation, wherein the formulation comprises ethylhexyl olivate, squalane, or squalene, and C13-15 alkane,

In several embodiments, there is provided for herein a topical formulation, wherein the formulation comprises ethylhexyl olivate, squalane, or squalene, and at least one saturated hydrocarbon with a chain length ranging from about 10 to about 20 carbons.

In several embodiments, the ethylhexyl olivate is present in an amount ranging between about 25% and about 80% (e.g., about 25, 30, 35, 40, 45, 50, 55, 60, 65, 70, 75, or 80%, including amounts between those listed). In several embodiments, the squalane or squalene is present in an amount ranging between about 0.1% and about 20% (e.g., about 0.1, 0.5, 1, 2, 3, 4, 5, 7, 9, 11, 13, 15, or 20%, including amounts between those listed). In several embodiments, the saturated hydrocarbon, such as C13-15 alkane, is present in an amount ranging between about 25% and about 80% (e.g., about 25, 30, 35, 40, 45, 50, 55, 60, 65, 70, 75, or 80%, including amounts between those listed).

In several embodiments, the ethylhexyl olivate is present in an amount ranging between 40 and 70% (e.g., about 40, 45, 50, 55, 60, 65, or 70%, including amounts between those listed). In several embodiments, the formulation does not comprise squalene, but squalane is present in an amount ranging from about 1% and about 20% (e.g., about 1, 2, 4, 5, 8, 10, 12, 14, 16, 18, or 20%, including amounts between those listed). In several embodiments, the C13-C15 alkane or other saturated hydrocarbon is present in an amount between about 25% to about 60% (e.g., about 25, 30, 35, 40, 45, 50, 55, or 60%, including amounts between those listed).

In several embodiments, the formulation is fragrance free. Optionally, in several embodiments, the formulation further comprises fragrance. In several embodiments, the formulation is a serum. In several embodiments, the formulation is configured to be applied to hair. In several embodiments, the formulation is safe for eczema-prone skin.

In several embodiments, there is provided a topical formulation, comprising water, sodium lauroyl methyl isethionate, acrylates copolymer, cocamidopropylamine oxide, wherein the cocamidopropylamine oxide is present in an amount between about 0.5% and about 5.0%; bisabolol, guar hydroxypropyltrimonium chloride, and optionally, at least two of phenoxyethanol, ethylhexylglycerin, citric acid and aminomethyl propanol.

In several embodiments, the water is present in an amount between about 60% to about 98% (including between about 65% and about 95%). In several embodiments, the sodium lauroyl methyl isethionate is present in an amount between about 1% and about 20% (including about 1-2%, about 2-3%, about 3-4%, about 5-6%, about 6-6%, about 7-10%, about 10-15%, or about 15-25%, including any amount between those listed). In several embodiments, the acrylates copolymer is present in an amount between about 0.1 and 7% (including between about 0.5% and about 5%, including between about 0.5-0.7%, about 0.7-0.9%, about 0.9-1.2%, about 1.2-1.5%, about 1.5-2.0%, about 2-4%, or about 4-7% including any amount between those listed). In several embodiments, the bisabolol is present in an amount between about 0.01% and about 1% (including about 0.01% about 0.07%, about 0.9%, about 0.1%, about 0.12%, about 0.15%, about 0.17%, about 0.2%, about 0.3%, about 0.5%, about 0.75%, and about 1.0, including any amount between those listed). In several embodiments, the guar hydroxypropyltrimonium chloride is present in an amount between 0.01% and 5% (including about 0.01% about 0.07%, about 0.9%, about 0.1%, about 0.12%, about 0.15%, about 0.17%, about 0.2%, about 0.23%, about 0.25%, about 0.275%, about 0.3%, about 0.5%, about 0.75%, and about 1.0, including any amount between those listed)

In several embodiments, there is provided a topical formulation, comprising water, sodium lauroyl methyl isethionate, acrylates copolymer, cocamidopropylamine oxide, bisabolol, and C13-C15 alkanes.

In several embodiments, the water is present in an amount between about 60% to about 98% (including between about 65% and about 95%). In several embodiments, the sodium lauroyl methyl isethionate is present in an amount between about 1% and about 20% (including about 1-2%, about 2-3%, about 3-4%, about 5-6%, about 6-6%, about 7-10%, about 10-15%, or about 15-25%, including any amount between those listed). In several embodiments, the acrylates copolymer is present in an amount between about 0.1 and 7% (including between about 0.5% and about 5%, including between about 0.5-0.7%, about 0.7-0.9%, about 0.9-1.2%, about 1.2-1.5%, about 1.5-2.0%, about 2-4%, or about 4-7% including any amount between those listed). In several embodiments, the bisabolol is present in an amount between about 0.01% and about 1% (including about 0.01% about 0.07%, about 0.9%, about 0.1%, about 0.12%, about 0.15%, about 0.17%, about 0.2%, about 0.3%, about 0.5%, about 0.75%, and about 1.0, including any amount between those listed). In several embodiments, the cocamidopropylamine oxide is present in an amount between about 0.25% and about 7% (including about 0.5% to about 5.0%; including about 0.25%, about 0.3%, about 0.4%, about 0.45%, about 0.5%, about 0.55%, about 0.6%, about 0.65%, about 0.7%, about 0.75%, about 0.8%, about 0.85%, about 0.9%, about 0.95%, about 1.0%, about 2.0%, about 3.0%, about 4.0%, about 5.0%, about 6.0%, about 7.0%, including any amount between those listed). In several embodiments, the bisabolol is present in an amount between about 0.01% and about 1% (including about 0.01% about 0.07%, about 0.9%, about 0.1%, about 0.12%, about 0.15%, about 0.17%, about 0.2%, about 0.3%, about 0.5%, about 0.75%, and about 1.0, including any amount between those listed). In several embodiments, the C13-C15 alkanes are present in an amount between 0.01% and 1% (including about 0.01%, 0.025%, about 0.05%, about 0.075%, about 0.1%, about 0.125%, about 0.15%, about 0.175%, about 0.2%, about 0.4%, about 0.6%, about 0.8%, or about 1.0%, including any amount between those listed).

In several embodiments, the sodium lauroyl methyl isethionate is present in an amount ranging from about 3% to about 10%; wherein the phenoxyethanol and ethylhexylglycerin (when present) are present in a combined amount of between about 0.5% and about 1.5%; and citric acid is present in an amount between about 0.001% and about 0.5%. In several embodiments, the formulation further comprises one or both of sodium hydroxide in an amount ranging between about 0.001% and about 0.01% and sodium benzoate in an amount ranging between about 0.001% and 0.5%.

In several embodiments, the formulation further comprising at least one neutralizer of metal ions comprising trisodium ethylenediamine disuccinate, wherein the trisodium ethylenediamine disuccinate is present in an amount ranging from about 0.01% to about 0.7%.

In several embodiments, the formulation further comprises squalane and/or squalene in an amount ranging between about 0.01% and about 0.7%.

In several embodiments, the topical formulation is formulated as a face wash, as a body wash, or as a shampoo.

In several embodiments, the topical formulation is fragrance free. However, in several embodiments, the formulation optionally further comprises fragrance. In several embodiments, the formulation is safe for eczema-prone skin.

There are also provided for herein methods of cleansing skin, comprising applying a formulation, or instructing the application of a formulation, according to the present disclosure to skin to be cleansed. In several embodiments, the skin is dry. In several embodiments, the skin is oily. In several embodiments, the skin has wrinkles. In several embodiments, the skin has inflammation. In several embodiments, the skin has blemishes. In several embodiments, the skin is sun damaged. In several embodiments, the skin is healthy. Use of a formulation as disclosed for the cleansing of skin and/or hair is provided for herein.

In several embodiments, there is provided a method of improving or maintaining the health, shine, strength, thickness, fullness, and/or volume of hair, comprising applying a formulation, or instructing the application of a formulation, according to embodiments disclosed herein. In several embodiments, there is provided a use of a formulation according to embodiments disclosed herein for improving or maintaining the health, shine, strength, thickness, fullness, and/or volume of hair. In several embodiments, a topical formulation, use, method, according to embodiments disclosed herein, the formulation excludes irritants. In several embodiments, the formulation excludes comedogenic elements having a Fulton scale grade greater than 2. In several embodiments, the topical formulation, use, method, according to embodiments disclosed herein, is safe for use on one or more of sensitive skin, acne-prone skin, and eczema prone-skin.

### DETAILED DESCRIPTION

Despite the large number of cosmetic formulations on the market to cleanse skin, there is a need for formulations that can cleanse, moisturize, treat, and/or prevent damaged skin in addition to offering anti-aging and nourishing effects on skin.

Several embodiments of the present invention relate to topical skin formulations for cleansing, hydrating, moisturizing skin and/or preventing and/or repairing skin conditions. The formulations described herein can be beneficial for both healthy and damaged skin, for example skin that is irritated through aging, lack of moisture, sun exposure, or other environmental or chemical stressors. In addition, many formulations also nourish the skin, improve overall appearance, texture, improvements in pore size, skin tone and/or firmness, either directly or indirectly (e.g., cleansed skin may exhibit increased firmness and/or reduced pore size). In several embodiments described herein, the formulations provide a rejuvenated and refreshed appearance after daily use for 1, 2, 3 weeks. Certain improvements in skin may be visible or felt within days of use.

In several embodiments, the formulations described herein are free from one or more (or all) of the following: parabens, sulfates, phthalates, fragrance (synthetic and/or natural, depending on the embodiment), EDTA, talc, dyes, mineral oils, drying alcohols, glycol, gluten, and chemical sunscreen. In several embodiments, the formulations are non-irritating and/or noncomedogenic. In several embodiments, the formulation is vegan and free from animal products (e.g., the components of the formulation are plant-based or synthetic).

The formulations described herein can provide both short-term and long-term benefits according to several embodiments. Beneficial effects from use of the formulations described herein occur upon use, within 1-2 days, about 7 days, about 14 days, or within about 4 weeks. In several embodiments, the skin appears improved after application of the formulation and benefits continue over about 2-6 weeks, about 6-12 weeks, about 12-24 weeks, or about 24-52 weeks of use. In several embodiments, long lasting effects on the skin are achieved in less than 1-3 months.

Several embodiments of the invention comprise a formulation for soothing irritated skin while also reinforcing the skin's natural defenses. In some embodiments, the formulation optionally includes ingredients that provide protection against UV damage. In some embodiments, the formulation is a liquid, serum, gel or cream. In some embodiments, the formulation is colorless (e.g., clear); however, in other embodiments, the formulation contains color. In some embodiments, the pH of the formulations is slightly basic, slightly acidic or neutral. In some embodiments, a pH of 3-5, 4-6, 5-7, 6-8, or ranges in between, are provided. Lower or higher pH values may also be used.

According to several embodiments, the formulations described herein can be dispensed through a pump attached to a container, dispensers mounted on wall or on a surface and/or poured directly from the container or a squeeze bottle. The formulations may be applied by hand, by sponge, by spraying, by dropper, or by brush or other applicator.

In some embodiments, after the formulation is applied to the skin, the formulation can be removed from the skin with water. In certain embodiments, the formulations can be removed without water, which can be accomplished by massaging the cleanser onto the skin, and, for example, wiping any excess cleanser with a dry or wet cloth, paper towel or facial tissue. In several embodiments, the formulations are meant to be applied to the skin, the hair, and/or skin and hair, but not purposefully removed (e.g., a “leave in” formulation).

Where numerical values are provided herein, including the tables below, those values may be for individual ingredients or for groups of ingredients. In some embodiments, the formulation comprises, consists essentially of or consists of several (e.g., 4, 6, 8, 10, 12, 15) or all of the following groups of ingredients listed in Table 1 below.

TABLE 1
Skin and Hair Care Formulations

| | | % total formula in some |
|---|---|---|
| | | embodiments as, for |
| | | example, % m/m, % m/v, |
| Group | Example ingredients in each group | % w/w, % w/v, % v/v |
| Group | One or more solvents such as water, siloxanes (e.g., | about 10-99% (e.g., about 10, |
| 1A | cyclopentasiloxane), caprylic/capric triglycerides, | 15, 25, 50, 60, 70, 80, 90, 95, |
| | glycerin and/or propanediol | 98, 99%, or any amount in |
| | | between) |
| Group | One or more neutralizing agents such as | about 0.1-2% (e.g., 0.1, 0.2, |
| 1B | aminomethyl propanol, aminodeoxygluticol, L- | 0.4, 0.5, 0.55, 0.575, 0.6, |
| | Proline, Glycoxylic acid and/or Ethanolamine | 0.75, 0.8, 0.85, 0.9, 0.95, 1, |
| | | 1.5, 2%, or any amount in |
| | | between) |
| Group | One or more agents for adjusting pH and/or | about 0.001-2% (e.g., about |
| 1C | chelation such as citric acid, sebacic acid, sodium | 0.001, 0.003, 0.005, 0.0075, |
| | phytate, sodium hydroxide, disodium | 0.01, 0.04, 0.05, 0.06, 0.08, |
| | Ethylenediaminetetraacetic acid (EDTA), trisodium | 0.1, 0.125, 0.15, 0.2, 0.25, |
| | ethylenediamine disuccinate and/or aminomethyl | 0.3, 0.32, 0.34, 0.36, 0.4, 0.45, |
| | propanol | 0.5, 0.55, 0.575, 0.6, 0.75, |
| | | 0.8, 0.85, 1, 1.5, 2%, or any |
| | | amount in between) |
| Group | One or more antioxidants with anti-inflammatory | about 0.0005-0.5% (e.g., |
| 1D | properties such as bisabolol, Vitamin E compounds | 0.0005, 0.00075, 0.001, 0.05, |
| | (e.g., tocopherol), Niacinamide and/or Vitamin A | 0.1, 0.15, 0.2, 0.25, 0.3, 0.4, |
| | compounds | 0.5%, or any amount in |
| | | between) |
| Group | One or more agents that act as a preservative and/or | about 0.01-2% (e.g., about |
| 1E | biocide such as phenoxyethanol, benzyl alcohol, | 0.01, 0.04, 0.05, 0.06, 0.08, |
| | sodium benzoate, ethylhexylglycerin, | 0.1, 0.125, 0.15, 0.2, 0.25, |
| | Behentrimonium chloride, citric acid, potassium | 0.3, 0.5, 0.75, 0.8, 0.9, 1, 1.5, |
| | sorbate, Caprylhydroxamic Acid and/or | 2%, or any amount in |
| | phytosphingosine | between) |
| Group | One or more agents that act as a stabilizer, | about 0.01-5% (e.g., 0.01, |
| 1F | suspending agents and/or thickener such as | 0.04, 0.05, 0.06, 0.08, 0.1, |
| | acrylates copolymer, acrylates crosspolymer, | 0.15, 0.2, 0.25, 0.3, 0.4, 0.45, |
| | sodium phytate, cocamidopropylamine oxide and/or | 0.5, 0.55, 0.75, 1, 1.5, 2, 2.25, |
| | xanthum gum | 2.5, 3, 4, 4.5, 4.75, 5%, or |
| | | any amount in between) |
| Group | One or more foaming agents such as | about 1-7% (e.g., 1, 1.25, 1.5, |
| 1G | cocamidopropylamine oxide | 2, 2.1, 2.4, 2.5, 3, 3.5, 4, 4.5, |
| | | 4.75, 5, 5.5, 6, 7%, or any |
| | | amount in between) |
| Group | One or more conditioning | about 0.05-4% (e.g., 0.05, |
| 1I | agents such as guar hydroxypropyltrimonium | 0.06, 0.07, 0.08, 0.09, 0.1, |
| | chloride, Behentrimonium chloride, and/or | 0.15, 0.2, 0.25, 0.3, 0.4, 0.5, |
| | sarcosine | 1, 1.5, 2, 2.25, 2.5, 3, 3.5, 4%, |
| | | or any amount in between) |
| Group | One or more emollients such as squalane (or | about 0.05-70% (e.g., 0.05, |
| 1J | optionally squalene), lauryl lactyl lactate, | 0.075, 0.09, 0.1, 0.15, 0.2, |
| | propanediol, lecithin, caprylic/capric triglyceride, | 0.3, 0.4, 0.5, 1, 2, 2.5, 3, 3.5, |
| | Glyceryl stearate, ethylhexyl olivate and/or C13-15 | 4, 5, 10, 20, 30, 35, 40, 45 |
| | alkane | 50, 55, 57, 59, 60, 70% or any |
| | | amount in between) |
| Group | One or more humectants such as caprylyl glycol, | about 0.005-5% (e.g., about |
| 1K | glycerin, propanediol, lecithin, squalane (or | 0.005, 0.0075, 0.01, 0.05, |
| | optionally squalene), cetearyl alcohol, shea butter | 0.07, 0.09, 0.1, 0.15, 0.2, |
| | cetyl esters and/or arginine | 0.25, 0.3, 0.5, 0.75, 1, 2, 2.5, |
| | | 3, 3.5, 4, 5%, or any amount |
| | | in between) |
| Group | One or more anti-static agents such as guar | about 0.1-5% (e.g., 0.1, 0.2, |
| 1L | hydroxypropyltrimonium chloride, | 0.25, 0.3, 0.5, 1, 2, 2.5, 3, 3.5, |
| | Stearamidopropyl dimethylamine, Behentrimonium | 4, 5% or any amount in |
| | chloride, polyquaternium-7, Cetrimonium Chloride, | between) |
| | and/or polyquaternium - 10 | |
| Group | One or more anti-dandruff agents such as | about 0.1-1% (e.g., about 0.1, |
| 1M | propanediol caprylate | 0.15, 0.2, 0.25, 0.3, 0.5, 0.75, |
| | | 1%, or any amount in |
| | | between) |
| Group | One or more surfactants such as sodium lauroyl | about 7.5-50% (e.g., 7.5, 8, 9, |
| 1N | methyl isethionate, lecithin, Glyceryl stearate, | 10, 11, 12, 13, 14, 15, 20, 25, |
| | sodium lauroyl oat amino acids, sodium lauroyl | 27.5, 30, 35, 37.5, 40, 45, |
| | apple amino acids, potassium laureth phosphate, | 50%, or any amount in |
| | Stearamidopropyl dimethylamine, and/or sodium | between) |
| | cocoyl glutamate | |
| Group | One or more ultraviolet (UV) absorbing and/or | about 0.1-1% (e.g., about 0.1, |
| 1O | blocking agents, such as avobenzone, homosalate, | 0.15, 0.2, 0.25, 0.3, 0.5, 0.75, |
| | zinc oxide and/or | 1%, or any amount in |
| | titanium dioxide, sunflower seed oil, caffeine, | between) |
| | prickly pear extract, jojoba oil, glycerin, castor oil, | |
| | raspberry seed extract, Morete (buriti) oil, Moringa | |
| | Oleifera seed extract | |

In some embodiments, the formulation comprises, consists essentially of or consists of several (e.g., 4, 6, 8, 10, 12) or all of the following groups of ingredients, or individual ingredients within a group, listed in Table 2 below:

TABLE 2
Cleansing Formulation #1

| | % total formula in some embodiments as, for |
|---|---|
| Ingredients | example, % m/m, % m/v, % w/w, % w/v, % v/v |
| Water/Aqua/Eau | about 60-85% (e.g., about 60, 65, 70, 72.5, 75, |
| | 77, 78, 78.2, 78.4, 78.6, 78.9, 79.5, 80, 82.5, 85 |
| | %, or any amount in between) |
| One or more of Cocamidopropylamine | about 0.05-4% (e.g., about 0.05, 0.1, 0.15, 0.2, |
| Oxide, Squalane, C13-15 Alkane, Bisabolol | 0.25, 0.3, 0.35, 0.4, 0.45, 0.5, 0.75, 1, 1.25, 1.5, |
| | 1.75, 2, 2.25, 2.5, 2.75, 3, 4%, or any amount in |
| | between) |
| One or more of Phenoxyethanol, | about 0.05-15% (e.g., about 0.05, 0.06, 0.08, |
| Ethylhexylglycerin, Sodium Lauroyl Methyl | 0.09, 0.1, 0.2, 0.3, 0.4, 0.5, 1, 1.5, 1.75, 2, 2.25, |
| Isethionate, Lauryl Lactyl Lactate, Acrylates | 2.5, 3, 3.2, 3.4, 3.6, 3.8, 4, 5, 6, 7.5, 8, 8.5, 9, 9.2, |
| Copolymer | 9.4, 9.6, 9.8, 10, 10.5, 11, 12, 14, 15%, or any |
| | amount in between) |
| One or more of Glycerin, Guar | about 0.1-6% (e.g., about 0.1, 0.15, 0.2, 0.25, |
| Hydroxypropyltrimonium Chloride | 0.3, 0.35, 0.4, 0.45, 0.5, 0.75, 1, 1.5, 2, 2.25, 2.5, |
| | 2.6, 2.7, 2.8, 2.9, 3, 3.2, 3.4, 3.6, 3.8, 4, 5, 6%, or |
| | any amount in between) |
| Tocopherol (or other antioxidant) | about 0.0001-0.5% (e.g., about 0.0001, 0.0005, |
| | 0.001, 0.004, 0.005, 0.01, 0.05, 0.1, 0.4, 0.5%, |
| | or any amount in between) |
| Sodium Benzoate | about 0.05-3% (e.g., about 0.05, 0.1, 0.15, 0.175, |
| | 0.2, 0.25, 0.3, 0.35, 0.4, 0.45, 0.5, 0.75, 1, 1.5, 2, |
| | 2.5, 3%, or any amount in between) |
| Trisodium Ethylenediamine Disuccinate | about 0.05-3% (e.g., about 0.05, 0.1, 0.15, 0.175, |
| | 0.2, 0.25, 0.3, 0.32, 0.34, 0.36, 0.38, 0.4, 0.45, |
| | 0.5, 0.75, 1, 1.5, 2, 2.5, 3%, or any amount in |
| | between) |
| Citric Acid | about 0.05-3% (e.g., about 0.05, 0.1, 0.125, 0.15, |
| | 0.175, 0.2, 0.25, 0.3, 0.35, 0.4, 0.45, 0.5, 0.75, 1, |
| | 1.5, 2, 2.5, 3%, or any amount in between) |
| Sodium Hydroxide | about 0.0001-0.5% (e.g., about 0.0001, 0.0005, |
| | 0.001, 0.002, 0.003, 0.004, 0.005, 0.01, 0.05, 0.1, |
| | 0.4, 0.5%, or any amount in between) |
| Aminomethyl Propanol | about 0.05-2% (e.g., about 0.05, 0.1, 0.2, 0.3, |
| | 0.4, 0.5, 0.6, 0.7, 0.75, 0.8, 0.85, 0.9, 1, 1.5, 2%, |
| | or any amount in between) |

In some embodiments, the formulation comprises, consists essentially of or consists of several (e.g., 4, 6, 8, 10, 12) or all of the following groups of ingredients, or individual ingredients within a group, listed in Table 3 below:

TABLE 3
Cleansing Formulation #2

| | % total formula in some embodiments as, for |
|---|---|
| Ingredients | example, % m/m, % m/v, % w/w, % w/v, % v/v |
| Water/Aqua/Eau | about 60-85% (e.g., about 60, 65, 70, 72.5, 75, |
| | 75.2, 75.4, 75.6, 75.8, 76, 76.5, 77, 78, 78.2, |
| | 78.4, 78.6, 78.9, 79.5, 80, 82.5, 85%, or any |
| | amount in between) |
| Acrylates Copolymer | about 1-6% (e.g., about 1, 1.1, 1.2, 1.25, 1.3, 1.4, |
| | 1.5, 1.75, 2, 2.25, 2.5, 2.6, 2.7, 2.8, 2.9, 3, 3.2, |
| | 3.4, 3.6, 3.8, 4, 5, 6%, or any amount in between) |
| One or more of C13-15 Alkanes, Bisabolol, | about 0.05-3% (e.g., about 0.05, 0.1, 0.15, 0.2, |
| Aminomethyl Propanol, Fragrance | 0.25, 0.3, 0.35, 0.4, 0.45, 0.5, 0.75, 1, 1.5, 2, 2.5, |
| | 3%, or any amount in between) |
| One or more of Phenoxyethanol, | about 0.05-20% (e.g., about 0.05, 0.06, 0.067, |
| Ethylhexylglycerin Polyquaternium-7, | 0.08, 0.09, 0.1, 0.15, 0.2, 0.25, 0.3, 0.35, 0.4, |
| Sodium Lauroyl Methyl Isethionate, | 0.45, 0.5, 0.75, 1, 1.5, 2, 2.5, 3, 5, 7, 8.5, 9, 9.2, |
| Cocamidopropylamine Oxide | 9.4, 9.6, 9.8, 10, 10.5, 11, 12, 13, 13.5, 14, 14.2, |
| | 14.4, 14.6, 14.7, 14.8, 15, 15.5, 16, 17, 18, 20%, |
| | or any amount in between) |
| Tocopherol | about 0.0001-0.5% (e.g., about 0.0001, 0.0005, |
| | 0.001, 0.004, 0.005, 0.01, 0.05, 0.1, 0.4, 0.5%, |
| | or any amount in between) |
| One or more of Glycerin, Guar | about 0.1-6% (e.g., about 0.1, 0.15, 0.2, 0.25, |
| Hydroxypropyltrimonium Chloride | 0.26, 0.28, 0.3, 0.35, 0.4, 0.45, 0.5, 0.75, 1, 1.5, |
| | 2, 2.5, 3, 4, 5, 6%, or any amount in between |
| Trisodium Ethylenediamine Disuccinate | about 0.05-3% (e.g., about 0.05, 0.1, 0.2, 0.3, |
| | 0.4, 0.45, 0.475, 0.5, 0.55, 0.6, 0.7, 0.75, 1, 1.5, |
| | 2, 2.5, 3%, or any amount in between |
| Sodium Benzoate | about 0.05-3% (e.g., about 0.05, 0.1, 0.15, 0.175, |
| | 0.2, 0.25, 0.3, 0.35, 0.4, 0.45, 0.5, 0.75, 1, 1.5, 2, |
| | 2.5, 3%, or any amount in between) |
| Citric Acid | about 0.05-3% (e.g., about 0.05, 0.1, 0.125, 0.15, |
| | 0.175, 0.2, 0.25, 0.3, 0.35, 0.4, 0.45, 0.5, 0.75, 1, |
| | 1.5, 2, 2.5, 3%, or any amount in between) |
| Disodium Ethylenediamine Disuccinate | about 0.05-3% (e.g., about 0.05, 0.1, 0.15, 0.175, |
| | 0.2, 0.25, 0.3, 0.32, 0.34, 0.36, 0.38, 0.4, 0.45, |
| | 0.5, 0.75, 1, 1.5, 2, 2.5, 3%, or any amount in |
| | between) |
| Sodium Hydroxide | about 0.0001-0.5% (e.g., about 0.0001, 0.0005, |
| | 0.001, 0.002, 0.003, 0.004, 0.005, 0.01, 0.05, 0.1, |
| | 0.4, 0.5%, or any amount in between) |

In some embodiments, the formulation comprises, consists essentially of or consists of several (e.g., 4, 6, 8, 10, 12) or all of the following groups of ingredients, or individual ingredients within a group, listed in Table 4 below:

TABLE 4
Cleansing Formulation #3

| | % total formula in some embodiments as, for |
|---|---|
| Ingredients | example, % m/m, % m/v, % w/w, % w/v, % v/v |
| Water/Aqua/Eau | about 60-95% (e.g., about 60, 65, 70, 72.5, 75, |
| | 77, 78, 78.2, 78.4, 78.6, 78.9, 79.5, 80, 82.5, 85, |
| | 90, 90.2, 90.4, 90.6, 90.8, 90.9, 91, 92, 95%, or |
| | any amount in between) |
| One or more of Cocamidopropylamine | about 0.05-4% (e.g., about 0.05, 0.1, 0.15, 0.2, |
| Oxide, Squalane, C13-15 Alkane, Bisabolol | 0.25, 0.3, 0.35, 0.4, 0.45, 0.5, 0.75, 1, 1.25, 1.5, |
| | 1.75, 2, 2.25, 2.5, 2.75, 3, 4%, or any amount in |
| | between) |
| One or more of Phenoxyethanol, | about 0.01-15% (e.g., about 0.01, 0.05, 0.06, |
| Ethylhexylglycerin, Sodium Lauroyl | 0.08, 0.09, 0.1, 0.2, 0.3, 0.4, 0.5, 1, 1.5, 1.75, 2, |
| Methyl Isethionate, Acrylates Copolymer | 2.25, 2.5, 3, 3.2, 3.4, 3.6, 3.8, 4, 5, 6, 7.5, 8, 8.5, |
| | 9, 9.2, 9.4, 9.6, 9.8, 10, 10.5, 11, 12, 14, 15%, or |
| | any amount in between) |
| Guar Hydroxypropyltrimonium Chloride | about 0.1-6% (e.g., about 0.1, 0.15, 0.2, 0.25, |
| | 0.3, 0.35, 0.4, 0.45, 0.5, 0.75, 1, 1.5, 2, 2.25, 2.5, |
| | 2.6, 2.7, 2.8, 2.9, 3, 3.2, 3.4, 3.6, 3.8, 4, 5, 6%, or |
| | any amount in between) |
| Tocopherol (or other antioxidant) | about 0.0001-0.5% (e.g., about 0.0001, 0.0005, |
| | 0.001, 0.004, 0.005, 0.01, 0.05, 0.1, 0.4, 0.5%, |
| | or any amount in between) |
| Sodium Benzoate | about 0.05-3% (e.g., about 0.05, 0.1, 0.15, 0.175, |
| | 0.2, 0.25, 0.3, 0.35, 0.4, 0.45, 0.5, 0.75, 1, 1.5, 2, |
| | 2.5, 3%, or any amount in between) |
| Trisodium Ethylenediamine Disuccinate | about 0.05-3% (e.g., about 0.05, 0.1, 0.15, 0.175, |
| | 0.2, 0.25, 0.3, 0.32, 0.34, 0.36, 0.38, 0.4, 0.45, |
| | 0.5, 0.75, 1, 1.5, 2, 2.5, 3%, or any amount in |
| | between) |
| Citric Acid | about 0.01-3% (e.g., about 0.01, 0.05, 0.1, 0.125, |
| | 0.15, 0.175, 0.2, 0.25, 0.3, 0.35, 0.4, 0.45, 0.5, |
| | 0.75, 1, 1.5, 2, 2.5, 3%, or any amount in |
| | between) |
| Aminomethyl Propanol | about 0.05-2% (e.g., about 0.05, 0.1, 0.2, 0.3, |
| | 0.4, 0.5, 0.6, 0.7, 0.75, 0.8, 0.85, 0.9, 1, 1.5, 2%, |
| | or any amount in between) |

In several embodiments, the formulations according to any of Tables 1-4 comprise a combination of various combination groups and individual ingredients.

In some embodiments, formulations according to any of Tables 1-4 have a pH at 25 degrees Celsius of about 5.5-7.7 (e.g., 6-7).

In the tables above, the ranges provided in the parentheticals include the overlapping ranges between the numbers. For example, a range of 10-50% (e.g., about 15, 25, 30, 35, 40, 45, 50%) includes, for example, ranges between 15-25%, 15-35%, 30-45%, 40-50%, etc., as well as the endpoints of the listed ranges. In one embodiment, the percentages provided are % m/m or % w/w.

In one embodiment, the formulation is configured for treating skin through use as a cleansing wash, for example a hydrating and/or a moisturizing cleanser for the face. In other embodiments, the formulation can be used as a body and/or scalp cleanser, or shampoo.

In one embodiment, the formulation comprises or consists essentially of water, sodium lauroyl methyl isethionate, glycerin, acrylates copolymer, guar hydroxypropyltrimonium chloride, cocamidopropylamine oxide, phenoxyethanol, ethylhexylglycerin and one or more ingredients selected from the group consisting of citric acid, lauryl lactyl lactate, squalane, C13-C15 alkane, bisabolol, aminomethyl propanol.

In one embodiment, the formulation comprises or consists essentially of a solvent, a coconut derived surfactant, a vegetable derived humectant, a synthetic copolymer, a guar gum derived anti-static agent, a coconut derived foaming agent, one or more of preservatives and/or biocides, emollients, viscosity increasing/decreasing agents, surfactants, conditioners, emulsifiers, fragrances, and pH adjusters. In some embodiments, the preservative is an antimicrobial. In some embodiments, the preservative is an antibacterial. In some embodiments, the preservative is an antifungal.

In several embodiments, the formulation comprises an oil-attracting and/or mild anionic surfactant. In several embodiments, the formulation comprises a lauric-acid derived surfactant. In several embodiments, the formulation comprises sodium lauroyl methyl isethionate. In several embodiments, the formulation comprises sodium cocoyl isethionate. In several embodiments, the formulation comprises sodium lauryl sulfate. In several embodiments, the combinations of such surfactants are used. In several embodiments, the formulation comprises a synthetic humectant. In several embodiments, the formulation comprises a fruit or plant derived humectant. In several embodiments, the formulation comprises one or more of propanediol, pentylene glycol BIO, beet sugar extract, sodium lactate, hydroxypropyltrimonium honey, or a salt of pyroglutamic acid, such as a sodium salt of pyroglutamic acid. In several embodiments, the combinations of such humectants are used. In several embodiments, the formulation comprises a semi-synthetic polymer or copolymer. In several embodiments, the formulation comprises a natural polymer or copolymer. In several embodiments, the formulation comprises polyglutamic acid. In several embodiments, the formulation comprises a combination of acrylic acid and methacryluic acid monomers. In several embodiments, the formulation comprises one or more of xantham gums, celluloses starches, other gums, or other polysaccharides or complex carbohydrates. In several embodiments, combinations of such polymers are used. In several embodiments, the formulation comprises an anti-static and/or a conditioning agent. In several embodiments, the formulation comprises a plant derived anti-static and/or a conditioning agent. In several embodiments, the formulation comprises a synthetic anti-static and/or a conditioning agent. In some embodiments, the formulation comprises a combination of plant derived or synthetic anti-static and/or a conditioning agent. In some embodiments, the formulation comprises a stabilizer, suspending and/or thickening agent. In several embodiments, the formulation comprises a fruit or plant derived stabilizer, suspending and/or thickening agent. In several embodiments, the formulation comprises a nut derived stabilizer, suspending and/or thickening agent. In several embodiments, the formulation comprises cocamidopropyl betaine. In several embodiments, the formulation comprises a preservative and/or a biocide. In several embodiments, the formulation comprises a plant derived preservative and/or a biocide. In several embodiments, the formulation comprises a synthetic preservative and/or a biocide. In several embodiments, the formulation comprises phenoxyethanol, benzyl alcohol, sodium benzoate and/or ethylhexylglycerin. In several embodiments, combinations of such preservatives and/or biocides are used. In several embodiments, the formulation comprises an emollient. In several embodiments, the formulation comprises a plant and/or fruit derived emollient. In several embodiments, the formulation comprises a nut derived emollient. In several embodiments, the formulation comprises an animal derived emollient. In several embodiments, the formulation comprises one or more of shea butter, argan oil, jojoba oil, cocoa butter, lanolin, soline, chia seed oil or tamanu oil. In several embodiments, the formulation comprises one or more of cococaprylate caprate, butylene glycol dicaprylate dicaprate. In several embodiments, the formulation comprises an agent for adjusting pH and/or chelation. In several embodiments, the formulation comprises fruit derived agent for adjusting pH and/or chelation. In several embodiments, the formulation comprises synthetic agent for adjusting pH and/or chelation. In several embodiments, the formulation comprises lactic acid, aminomethyl propanol, propylene glycol ortriethanolamine. In several embodiments, the formulation comprises antioxidants with anti-inflammatory properties. In several embodiments, the formulation comprises antioxidants with anti-inflammatory properties derived from plants. In several embodiments, the formulation comprises synthetic antioxidants with anti-inflammatory properties.

In one embodiment, the formulation comprises or consists essentially of (i) water in the range of about 60-90% of the total formulation, (ii) sodium lauroyl methyl isethionate in the range of about 5-15% of the total formulation, (iii) acrylates copolymer in the range of about 1-5% of the total formulation and (iv) cocamidopropylamine oxide in the range of about 0.5-5% of the total formulation and (v) at least four or all of guar hydroxypropyltrimonium chloride, glycerin, C13-C15 alkane, aminomethyl propanol, bisabolol, collectively in the range of about 1%-6% of the total formulation, and (vi) one or more preservatives, viscosity increasing/decreasing agents, surfactants, conditioners, emulsifiers, fragrances, and pH adjusters, collectively in a range of about 1-10% of the total formulation.

In one embodiment, the formulation comprises or consists essentially of water in the range of 60-85% (e.g., about 70-85%), glycerin in the range of 0.05-8% (e.g., about 1-5%%), guar hydroxypropyltrimonium chloride in the range of about 0.01-3% (e.g., about 0.05-2.5%), citric acid in the range of about 0.05-2% (e.g., about 0.07-1.5%), phenoxyethanol and/or ethylhexylglycerin in a collective or individual range of about 0.01-2.5% (e.g., 0.05-2%), sodium lauroyl methyl isethionate in the range of about 3-20% (e.g., about 5-17%), lauryl lactyl lactate in the range of about 0.1-4% (e.g., about 0.5-3%), acrylates copolymer in the range of about 0.1-10% (e.g., about 0.5-5%), cocamidopropylamine oxide in the range of about 0.05-5% (e.g., about 0.1-4%), squalane in the range of about 0.05-5% (e.g., about 0.1-3%), C13-C15 alkane in the range of about 0.05-4% (e.g., about 0.08-2.5%), bisabolol in the range of about 0.01-4% (e.g., about 0.05-2.5%) and aminomethyl propanol in the range of about 0.05-4% (e.g., about 0.1-1.5%). In some embodiments, the ratio between any two of the disclosed components is 1:1, 1:2, 1:3, 1:4, 1:5, 1:6, 1:10, 1:15, 1:20, 1:50, 1:100, 1:200, 1:500, 1:1000, 1:5000, 1:10000 and in ranges in between.

In one embodiment, the formulation comprises or consists essentially of water in the range of 65-80% (e.g., about 75-85%), glycerin in the range of 0.05-5% (e.g., about 2-4%%), guar hydroxypropyltrimonium chloride in the range of about 0.05-2% (e.g., about 0.1-1.5%), and citric acid in the range of about 0.05-2% (e.g., about 0.1-1.25%).

Some embodiments, further comprise sodium lauroyl methyl isethionate in the range of about 5-20% (e.g., about 7-15%) and/or lauryl lactyl lactate in the range of about 0.5-2% (e.g., about 0.75-1.5%).

Several embodiments, further comprise aminomethyl propanol in the range of about 0.5-2% (e.g., about 0.6-1.2%).

Several embodiments further comprise one or both of phenoxyethanol (in the range of about 0.1-1.5% (e.g., 0.5-1.2%)) and ethylhexylglycerin (in the range of about 0.01-2% (e.g., about 0.05-1.5%)).

Several embodiments of the formulation further comprise bisabolol in the range of about 0.05-2% (e.g., about 0.1-1.5%).

Depending on the embodiment, the formulation optionally further comprises acrylates copolymer in the range of about 1-10% (e.g., about 1-5%). Also, some embodiments, comprise one or all of cocamidopropylamine oxide in the range of about 0.5-5% (e.g., about 0.5-4%), squalane in the range of about 0.05-2% (e.g., about 0.1-1.5%), and/or C13-C15 alkane (or hydrocarbon of another chain length) in the range of about 0.05-2% (e.g., about 0.1-1.5%).

In one embodiment, the formulation comprises or consists essentially of water in the range of 65-80% (e.g., about 75-85%), glycerin in the range of 0.05-5% (e.g., about 2-4%%), one or more conditioning agents in the range of about 0.05-2% (e.g., about 0.1-1.5%), one or more pH modifiers, in the range of about 0.05-2% (e.g., about 0.1-1.25%), one or more preservatives in the range of about 0.05-2% (e.g., about 0.05-1.5%), one or more surfactants in the range of about 5-20% (e.g., about 7-15%), one or more emollients in the range of about 0.5-2% (e.g., about 0.75-1.5%), one or more stabilizing and/or suspending agents in the range of about 1-10% (e.g., about 1-5%), one or more foaming agents in the range of about 0.5-5% (e.g., about 0.5-4%), one or more moisturizers in the range of about 0.05-2% (e.g., about 0.1-1.5%), one or more emollients such as an alkane in the range of about 0.05-2% (e.g., about 0.1-1.5%), one or more antioxidants with anti-inflammatory properties in the range of about 0.05-2% (e.g., about 01-1.5%) and one or more neutralizing agents in the range of about 0.5-2% (e.g., about 0.6-1.2%).

In one embodiment, the formulation is configured for treatment as a cleansing wash for the body. In other embodiments, the formulation can be used as a face and/or scalp cleanser. In several embodiments, the cleanser additionally provides hydrating and/or moisturizing effects.

In one embodiment, the formulation comprises or consists essentially of water, disodium EDTA, sodium lauroyl methyl isethionate, acrylates copolymer, cocamidopropylamine oxide, polyquaternium-7, glycerin, guar hydroxypropyltrimonium chloride and one or more ingredients selected from the group consisting of citric acid, phenoxyethanol, ethylhexylglycerin, C13-C15 alkane, bisabolol and aminomethyl propanol. In several embodiments, EDTA is replaced with an alternative chelating agent. In several embodiments, sodium phytate is used in addition to, or in place of, EDTA.

In one embodiment, the formulation comprises or consists essentially of a solvent, a coconut derived surfactant, a synthetic surfactant, a vegetable derived humectant, a synthetic copolymer, a guar gum derived anti-static agent, a coconut derived foaming agent, one or more of preservatives and/or biocides, emollients, viscosity increasing/decreasing agents, surfactants, conditioners, emulsifiers, fragrances, and pH adjusters. In some embodiments, the preservative is an antimicrobial. In some embodiments, the preservative is an antibacterial. In some embodiments, the preservative is an antifungal.

In several embodiments, the formulation comprises an oil-attracting and/or mild anionic surfactant. In several embodiments, the formulation comprises a lauric-acid derived surfactant. In several embodiments, the formulation comprises sodium lauroyl methyl isethionate. In several embodiments, the formulation comprises sodium cocoyl isethionate. In several embodiments, the formulation comprises sodium lauryl sulfate. In several embodiments, the combinations of such surfactants are used. In several embodiments, the formulation comprises a synthetic humectant. In several embodiments, the formulation comprises a fruit or plant derived humectant. In several embodiments, the formulation comprises one or more of propanediol, pentylene glycol BIO, beet sugar extract, sodium lactate, hydroxypropyltrimonium honey, or a salt of pyroglutamic acid, such as a sodium salt of pyroglutamic acid. In several embodiments, the combinations of such humectants are used. In several embodiments, the formulation comprises a semi-synthetic polymer or copolymer. In several embodiments, the formulation comprises a natural polymer or copolymer. In several embodiments, the formulation comprises polyglutamic acid. In several embodiments, the formulation comprises a combination of acrylic acid and methacryluic acid monomers. In several embodiments, the formulation comprises acrylamide monomers. In several embodiments, the formulation comprises one or more of xantham gums, celluloses starches, other gums, or other polysaccharides or complex carbohydrates. In several embodiments, combinations of such polymers are used. In several embodiments, the formulation comprises an anti-static and/or a conditioning agent. In several embodiments, the formulation comprises a plant derived anti-static and/or a conditioning agent. In several embodiments, the formulation comprises a synthetic anti-static and/or a conditioning agent. In some embodiments, the formulation comprises a combination of plant derived or synthetic anti-static and/or a conditioning agent. In some embodiments, the formulation comprises a stabilizer, suspending and/or thickening agent. In several embodiments, the formulation comprises a fruit or plant derived stabilizer, suspending and/or thickening agent. In several embodiments, the formulation comprises a nut derived stabilizer, suspending and/or thickening agent. In several embodiments, the formulation comprises cocamidopropyl betaine. In several embodiments, the formulation comprises a preservative and/or a biocide. In several embodiments, the formulation comprises a plant derived preservative and/or a biocide. In several embodiments, the formulation comprises a synthetic preservative and/or a biocide. In several embodiments, the formulation comprises phenoxyethanol, benzyl alcohol, sodium benzoate and/or ethylhexylglycerin. In several embodiments, combinations of such preservatives and/or biocides are used. In several embodiments, the formulation comprises an emollient. In several embodiments, the formulation comprises a plant and/or fruit derived emollient. In several embodiments, the formulation comprises a nut derived emollient. In several embodiments, the formulation comprises an animal derived emollient. In several embodiments, the formulation comprises one or more of shea butter, argan oil, jojoba oil, cocoa butter, lanolin, soline, chia seed oil or tamanu oil. In several embodiments, the formulation comprises one or more of cococaprylate caprate, butylene glycol dicaprylate dicaprate. In several embodiments, the formulation comprises an agent for adjusting pH and/or chelation. In several embodiments, the formulation comprises fruit derived agent for adjusting pH and/or chelation. In several embodiments, the formulation comprises synthetic agent for adjusting pH and/or chelation. In several embodiments, the formulation comprises lactic acid, aminomethyl propanol, propylene glycol ortriethanolamine. In several embodiments, the formulation comprises antioxidants with anti-inflammatory properties. In several embodiments, the formulation comprises antioxidants with anti-inflammatory properties derived from plants. In several embodiments, the formulation comprises synthetic antioxidants with anti-inflammatory properties.

In one embodiment, the formulation comprises or consists essentially of (i) water in the range of about 65-90% of the total formulation, (ii) sodium lauroyl methyl isethionate in the range of about 5-20% of the total formulation, (iii) acrylates copolymer in the range of about 0.5-5% of the total formulation and (iv) cocamidopropylamine oxide in the range of about 0.5-7% of the total formulation, (v) polyquaternium-7 in the range of about 0.05-3% of the total formulation and (vi) at least five or all of disodium EDTA, guar hydroxypropyltrimonium chloride, glycerin, C13-C15 alkane, aminomethyl propanol, bisabolol, collectively in the range of about 1-4% of the total formulation, and (vi) one or more preservatives, conditioners, viscosity increasing/decreasing agents, surfactants, emulsifiers, fragrances, colorants, and pH adjusters, collectively in a range of about 1-4% of the total formulation.

In one embodiment, the formulation comprises or consists essentially of water in the range of 60-90% (e.g., about 67-85%), disodium EDTA, or other chelating agent, in the range of about 0.01-2% (e.g., about 0.05-1%), glycerin in the range of 0.05-5% (e.g., about 1-4%), guar hydroxypropyltrimonium chloride in the range of about 0.01-2% (e.g., about 0.05-1.9%), citric acid in the range of about 0.01-2% (e.g., about 0.05-1.0%), phenoxyethanol and/or ethylhexylglycerin in a collective or individual range about 0.01-2.5% (e.g., 0.05-2%), polyquaternium-7 in the range of about 0.01-3% (e.g., about 0.05-2%), sodium lauroyl methyl isethionate in the range of about 5-25% (e.g., about 7-20%), acrylates copolymer in the range of about 0.05-5% (e.g., about 0.1-3%), cocamidopropylamine oxide in the range of about 0.05-7% (e.g., about 0.5-5%), C13-C15 alkane (other hydrocarbon of other chain length) in the range of about 0.01-5% (e.g., about 0.05-2%), bisabolol in the range of about 0.05-2% (e.g., about 0.075-1.5%), aminomethyl propanol in the range of about 0.05-2.5% (e.g., about 0.1-2%) and, optionally, fragrance in the range of about 0.05-2% (e.g., about 0.1-1.5%). In some embodiments, the ratio between any two of the disclosed components is 1:1, 1:2, 1:3, 1:4, 1:5, 1:6, 1:10, 1:15, 1:20, 1:50, 1:100, 1:200, 1:500, 1:1000, 1:5000, 1:10000 and in ranges in between.

In one embodiment, the formulation comprises or consists essentially of water in the range of 65-90% (e.g., about 67-85%), disodium EDTA in the range of about 0.01-1% (e.g., about 0.05-0.5%), glycerin in the range of 0.05-5% (e.g., about 1-3%), and sodium lauroyl methyl isethionate in the range of about 5-20% (e.g., about 7-20%%).

In several embodiments, the formulation further comprises guar hydroxypropyltrimonium chloride in the range of about 0.05-2% (e.g., about 0.1-1.5%) and optionally, citric acid in the range of about 0.05-2% (e.g., about 0.1-1.0%).

Several embodiments also employ bisabolol, depending on the embodiment, in the range of about 0.05-2% (e.g., about 0.075-1.0%). In several embodiments, aminomethyl propanol is included in the formulations, for example, in the range of about 0.05-2% (e.g., about 0.1-1.2%).

C13-C15 alkane, or a hydrocarbon of another chain length, is included in the formulation, in some embodiments, for example in the range of about 0.05-2% (e.g., about 0.1-1.2%).

Several embodiments further comprise one or both of acrylates copolymer (in the range of about 0.5-5% (e.g., about 0.75-3%)) and cocamidopropylamine oxide (in the range of about 0.5-7% (e.g., about 0.5-4%)).

Several embodiments, further comprise phenoxyethanol in the range of about 0.1-1.5% (e.g., 0.5-1.2%) and/or ethylhexylglycerin in the range of about 0.01-2% (e.g., about 0.05-1.5%). Depending on the embodiment, the formulation may further comprise polyquaternium-7 in the range of about 0.05-3% (e.g., about 0.1-2%).

Depending on the embodiment, optionally, fragrance is included in the formulation in the range of about 0.05-2% (e.g., about 0.1-1.5%).

In one embodiment, the formulation comprises or consists essentially of water in the range of 65-90% (e.g., about 67-85%), one or more chelating agents in the range of about 0.01-1% (e.g., about 0.05-0.5%), glycerin in the range of 0.05-5% (e.g., about 1-3%), one or more conditioning agents in the range of about 0.05-2% (e.g., about 0.1-1.5%), one or more pH modifiers in the range of about 0.05-2% (e.g., about 0.1-1.0%), one or more preservatives in the range of about 0.05-2% (e.g., about 0.75-1.5%), one or more neutralizing polymers in the range of about 0.05-3% (e.g., about 0.1-2%), one or more surfactants in the range of about 5-20% (e.g., about 7-20%%), one or more stabilizing and/or suspending agents in the range of about 0.5-5% (e.g., about 0.75-3%), one or more foaming agents in the range of about 0.5-7% (e.g., about 0.5-4%), one or more emollients such as alkane in the range of about 0.05-2% (e.g., about 0.1-1.2%), one or more antioxidants with anti-inflammatory properties in the range of about 0.05-2% (e.g., about 0.075-1.0%), one or more neutralizing agents in the range of about 0.05-2% (e.g., about 0.1-1.2%) and fragrance in the range of about 0.05-2% (e.g., about 0.1-1.5%).

In one embodiment, the formulation is configured for treatment as a cleansing wash for the body. In other embodiments, the formulation can be used as a face and/or scalp cleanser. In several embodiments, the cleanser additionally provides hydrating and/or moisturizing effects. In one embodiment, the formulation is configured for treatment as a cleansing wash, for example a hydrating and/or a moisturizing cleanser for infants, toddlers, adolescents, teens and/or young adults. In several embodiments, such formulations are configured to reduce or eliminate irritation of the eyes of the user, should the formulation contact the eye during use. In other embodiments, the formulation can be used as a cleansing wash for adults.

In one embodiment, the formulation comprises or consists essentially of water, sodium lauroyl methyl isethionate, guar hydroxypropyltrimonium chloride, cocamidopropylamine oxide, glycerin, trisodium ethylenediamine disuccinate, acrylates copolymer, C13-C15 alkane and one or more ingredients selected from the group of tocopherol, bisabolol, sodium benzoate, phenoxyethanol, ethylhexylglycerin, citric acid and aminomethyl propanol.

In one embodiment, the formulation comprises or consists essentially of a solvent, a coconut derived surfactant, a synthetic surfactant, a vegetable derived humectant, a synthetic copolymer, a guar gum derived anti-static agent, a coconut derived foaming agent, one or more of preservatives and/or biocides, emollients, viscosity increasing/decreasing agents, surfactants, conditioners, emulsifiers, fragrances, and pH adjusters. In some embodiments, the preservative is an antimicrobial. In some embodiments, the preservative is antibacterial. In some embodiments, the preservative is an antifungal.

In several embodiments, the formulation comprises an oil-attracting and/or mild anionic surfactant. In several embodiments, the formulation comprises a lauric-acid derived surfactant. In several embodiments, the formulation comprises sodium lauroyl methyl isethionate. In several embodiments, the formulation comprises sodium cocoyl isethionate. In several embodiments, the formulation comprises sodium lauryl sulfate. In several embodiments, the combinations of such surfactants are used. In several embodiments, the formulation comprises a synthetic humectant. In several embodiments, the formulation comprises a fruit or plant derived humectant. In several embodiments, the formulation comprises one or more of propanediol, pentylene glycol BIO, beet sugar extract, sodium lactate, hydroxypropyltrimonium honey, or a salt of pyroglutamic acid, such as a sodium salt of pyroglutamic acid. In several embodiments, the combinations of such humectants are used. In several embodiments, the formulation comprises a semi-synthetic polymer or copolymer. In several embodiments, the formulation comprises a natural polymer or copolymer. In several embodiments, the formulation comprises polyglutamic acid. In several embodiments, the formulation comprises a combination of acrylic acid and methacryluic acid monomers. In several embodiments, the formulation comprises acrylamide monomers. In several embodiments, the formulation comprises one or more of xantham gums, celluloses starches, other gums, or other polysaccharides or complex carbohydrates. In several embodiments, combinations of such polymers are used. In several embodiments, the formulation comprises an anti-static and/or a conditioning agent. In several embodiments, the formulation comprises a plant derived anti-static and/or a conditioning agent. In several embodiments, the formulation comprises a synthetic anti-static and/or a conditioning agent. In some embodiments, the formulation comprises a combination of plant derived or synthetic anti-static and/or a conditioning agent. In some embodiments, the formulation comprises a stabilizer, suspending and/or thickening agent. In several embodiments, the formulation comprises a fruit or plant derived stabilizer, suspending and/or thickening agent. In several embodiments, the formulation comprises a nut derived stabilizer, suspending and/or thickening agent. In several embodiments, the formulation comprises cocamidopropyl betaine. In several embodiments, the formulation comprises a preservative and/or a biocide. In several embodiments, the formulation comprises a plant derived preservative and/or a biocide. In several embodiments, the formulation comprises a synthetic preservative and/or a biocide. In several embodiments, the formulation comprises phenoxyethanol, benzyl alcohol, sodium benzoate and/or ethylhexylglycerin. In several embodiments, combinations of such preservatives and/or biocides are used. In several embodiments, the formulation comprises an emollient. In several embodiments, the formulation comprises a plant and/or fruit derived emollient. In several embodiments, the formulation comprises a nut derived emollient. In several embodiments, the formulation comprises an animal derived emollient. In several embodiments, the formulation comprises one or more of shea butter, argan oil, jojoba oil, cocoa butter, lanolin, soline, chia seed oil or tamanu oil. In several embodiments, the formulation comprises one or more of cococaprylate caprate, butylene glycol dicaprylate dicaprate. In several embodiments, the formulation comprises an agent for adjusting pH and/or chelation. In several embodiments, the formulation comprises fruit derived agent for adjusting pH and/or chelation. In several embodiments, the formulation comprises synthetic agent for adjusting pH and/or chelation. In several embodiments, the formulation comprises lactic acid, aminomethyl propanol, propylene glycol ortriethanolamine. In several embodiments, the formulation comprises antioxidants with anti-inflammatory properties. In several embodiments, the formulation comprises antioxidants with anti-inflammatory properties derived from plants. In several embodiments, the formulation comprises synthetic antioxidants with anti-inflammatory properties.

In one embodiment, the formulation comprises or consists essentially of (i) water in the range of about 70-95% of the total formulation, (ii) sodium lauroyl methyl isethionate, in the range of about 1-10% of the total formulation, (iii) acrylates copolymer in the range of about 0.1-4% of the total formulation, (iv) cocamidopropylamine oxide in the range of about 0.1-3% of the total formulation, (v) guar hydroxypropyltrimonium chloride in the range of about 0.05-4% of the total formulation, and (vi) at least three or all of trisodium ethylenediamine disuccinate, citric acid and aminomethyl propanol collectively in the range of 0.1-3% of the total formulation and (vii) preservatives, emollients, viscosity increasing/decreasing agents, surfactants, emulsifiers, fragrances, colorants, and pH adjusters, collectively in a range of about 0.5-3% of the total formulation.

In one embodiment, the formulation comprises or consists essentially of water in the range of about 70-95% (e.g., about 80-95%) of the total formulation, sodium lauroyl methyl isethionate in the range of about 1-10% (e.g., about 3-10%) of the total formulation, acrylates copolymer in the range of about 0.1-4% (e.g., about 0.5-4%) of the total formulation, cocamidopropylamine oxide in the range of about 0.1-3% (e.g., about 0.5-3%) of the total formulation, guar hydroxypropyltrimonium chloride in the range of about 0.05-4% (e.g., about 0.1-4%) of the total formulation, trisodium ethylenediamine disuccinate in the range of about 0.05-5% (e.g., about 0.1-4%) of the total formulation, tocopherol in the range of about 0.001-2% (e.g., about 0.001-1%) of the total formulation, aminomethyl propanol in the range of about 0.05-4% (e.g., about 0.1-4%) of the total formulation, sodium benzoate in the range of 0.001-3% (e.g., about 0.001-2%) of the total formulation, bisabolol in the range of about 0.001-2% (e.g., about 0.005-1.5%), C13-C15 alkane in the range of about 0.001-2% (e.g., about 0.005-1.5%), citric acid in the range of about 0.005-2% (e.g., about 0.005-1.5%), phenoxyethanol and/or ethylhexylglycerin in a collective or individual range of about 0.001-2.5% (e.g., 0.1-2%). In some embodiments, the ratio between any two of the disclosed components is 1:1, 1:2, 1:3, 1:4, 1:5, 1:6, 1:10, 1:15, 1:20, 1:50, 1:100, 1:200, 1:500, 1:1000, 1:5000, 1:10000 and in ranges in between.

In several embodiments, the formulation comprises or consists essentially of guar hydroxypropyltrimonium chloride, cocamidopropylamine oxide, acrylates copolymer, bisabolol, sodium lauryl methyl isethionate, phenoxyethanol and ethylhexylglycerin.

In one embodiment, the formulation comprises or consists essentially of water in the range of about 70-95% (e.g., about 80-95%) of the total formulation, one or more surfactants in the range of about 1-10% (e.g., about 3-10%) of the total formulation, one or more of stabilizer, suspending agents and/or thickeners in the range of about 0.1-4% (e.g., about 0.5-4%) of the total formulation, one or more foaming agents in the range of about 0.1-3% (e.g., about 0.5-3%) of the total formulation, one or more conditioning agents in the range of about 0.05-4% (e.g., about 0.1-4%) of the total formulation, one or more agents for adjusting pH and/or chelation in the range of about 0.01-5% (e.g., about 0.01-4%) of the total formulation, one or more antioxidants with anti-inflammatory properties in the range of about 0.001-2% (e.g., about 0.001-1%) of the total formulation, aminomethyl propanol in the range of about 0.05-4% (e.g., about 0.1-4%) of the total formulation, one or more preservatives and/or biocides in the range of 0.001-3% (e.g., about 0.001-2%) of the total formulation, and one or more emollients in the range of about 0.001-2% (e.g., about 0.005-1.5%).

In several embodiments in which C13-15 alkane is included in the formulation, non-polar C15 hydrocarbon is obtained from sugarcane. C13-15 alkane is used in several embodiments as a non-volatile emollient.

In several embodiments, the water used in the formulation is one or more of distilled, deionized, and/or sea water.

In alternative embodiments, Squalene is used in addition to or in place of Squalane.

In several embodiments in which bisabolol is included in the formulation, it is derived from Candeia tree. Bisabolol is used as an anti-inflammatory agent. In addition, or in lieu of, bisabolol, one or more of the following is provided: Vitamin E compounds, Niacinamide and/or Vitamin A compounds.

In several embodiments, the formulation comprises a stabilizer, suspending agent and/or thickener. Acrylates copolymer can be used herein as a stabilizing or a suspending agent or a thickener. In several other embodiments, cocamidopropylamine oxide can be used as a thickener and stabilizer.

In several embodiments, the formulation comprises a neutralizing agent. Aminomethyl propanol can be used herein as a neutralizing agent. In several other embodiments, aminodeoxygluticol, L-Proline, Glycoxylic acid and/or Ethanolamine can be used as a neutralizing agent.

The agents, ingredients and compounds described herein may be modified natural substances (e.g., isolates, extracts, purified, processed, chemically modified, etc.) or synthetic substances. To Applicant's knowledge, several combinations of ingredients disclosed herein represent unique enhanced formulations that are not naturally occurring (e.g., not found in nature in such combination). Moreover, in several embodiments, the individual ingredients may be modified to be structurally and/or functionally different than the naturally occurring species, thereby resulting in markedly unique effects.

Although preservatives are provided in certain embodiments, the formulations described herein can be manufactured with reduced or no synthetic preservatives. In some embodiments, the formulations are free of one or more of the following: parabens, phthalates, sulfates, mineral oil, gluten, allergens, and irritants.

In some embodiments, the formulations described herein can be used for cleansing, hydrating and moisturizing the skin in a subject, wherein the topical formulation is a liquid (such as serum, lotion, liquid, oil or cream), a gel, a spray, a powder, a jelly, or a combination thereof. In some embodiments, the topical formulation is a solid (e.g., soap bar). In some embodiments, the subject is a mammal. In some embodiments, the subject is a human. In some embodiments, the subject is 0, 1, 2, 3, 4, 5, 6, 7, 8, 9, 10, 15, 20, 25, 30, 35, 40, 45, 50, 55, 60, 65, 70, 75, 80, 85, 90, 95, 100, 105 years old, or any of the ranges in between.

In addition, in some embodiments, at least one of the following ingredients is included: a skin conditioning agent, a solvent, a silicone, an emollient, a preservative, a thickener, an antioxidant, an anti-inflammatory agent, and an excipient. Colorants and fragrances may additionally be included. In some embodiments, the formulation further includes one or more of the following: amino acids, peptides, phospholipids, additional vitamins, growth factors, and additional anti-aging compounds. Surfactants, gelling agents, and pH balancers may also be included.

Formulations for Improving, Treating and/or Maintaining Hair and/or Scalp

Despite the large number of cosmetic formulations on the market to treat hair, there remains a need for a cosmetic formulation that can treat, improve and/or maintain the health of the hair, including preventing further damage, and/or treat the scalp skin underlying the hair. Both healthy and damaged hair and/or scalp skin can benefit from several of the formulations described herein. Damaged skin can include skin with hyperpigmentation, dry skin, oily skin, acne and/or dandruff as well as treating dry, flaky scalp and scalp itching.

An enhanced glow or radiance is achieved by several embodiments. Many formulations are not only therapeutic and protective, but also nourish the skin, increase hydration, and improve overall appearance, texture and firmness. Thus, several formulations of the invention are beneficial for skin that is aged, sun-damaged, wrinkled, lax, and/or blemished. Formulations according to several embodiments can be used anywhere on the body, and are especially beneficial for the face, neck, scalp (in particular the hair follicle), décolletage and hands, where signs of skin damage and aging may be particularly prominent.

In some embodiments, the invention comprises a topical formulation for treating and protecting scalp (or non-scalp) skin in a subject, wherein the topical formulation is a liquid (such as a serum, lotion, liquid primer, oil or cream), a gel, a spray, or a combination thereof. In some embodiments, the formulations are particularly useful to improve the health of the skin, such as non-scalp skin, of a user, for example, after a dermatological procedure (including but not limited to brow lifts, blepharoplasty, botulin and other neurotoxins, facials, fillers, dermabrasion, microdermabrasion, micro-needling, peels, exfoliations, suctioning, fluid delivery, acid treatments, massage, extractions, energy-based and other treatments, such as lasers, thermal, radiofrequency, light (e.g., photofacials/IPL), etc.). In such embodiments, the skin of the user may be particularly sensitive to use of formulations that, unlike those provided for herein, are specifically designed to nourish and improve the health of the skin without irritation.

In some embodiments, the invention comprises a topical formulation for treating dry, flaky, and/or itchy scalp, hydrating and moisturizing the scalp skin in a subject, wherein the topical formulation is a liquid (such as serum, lotion, liquid, oil or cream), a gel, a spray, a powder, a jelly, or a combination thereof.

In some embodiments, the invention also provides a method of treating scalp disorders such as scalp irritation, scalp folliculitis, dandruff, psoriasis, eczema and/or itching comprising applying the formulation on the scalp.

In several embodiments, use of the formulations described herein reduces skin damage by about 10-100% (e.g., about 10%, 25%, 50%, 75%, 100% and ranges in between) after use. For example, significant improvements in non-scalp skin pore size, fine lines, overall appearance, radiance, skin smoothness, and/or skin tone (evenness), and/or improved scalp health (reduced itchiness, flaking etc.) are visible in several embodiments after 2 weeks, 4 weeks, 8 weeks or 12 weeks. Certain improvements in scalp and non-scalp skin and may be visible or felt within days of use. In several embodiments, the formulations provided for herein reduce and/or ameliorate one or more symptoms of irritation of skin (including itching or burning) and or reduce one or more symptoms of eczema. In several embodiments, the strength, thickness, shine, and/or body of hair to which the formulation has been applied show improvement. In several embodiments, hair loss is reduced after use of the formulations provided for herein.

In several embodiments, use of the formulations described herein promote hair growth and/or promote hair follicle development. In several embodiments, the formulations described herein can be used to strengthen the hair and/or improve the general condition of hair, including, but not limited to increasing hair density, thickening the hair, and/or increasing hair volume.

In some embodiments, the formulations described herein can be used for hair loss treatment. In several embodiments, the formulations alleviate or ameliorate androgenic alopecia, whether in males or females. In several embodiments, the formulations alleviate or ameliorate female-pattern hair loss in pre- and/or post-menopausal women. In some embodiments, the formulations can be used to reduce hair weakness and reduce structural damage to the hair.

In some embodiments, the formulations are used in the treatment of damaged hair. In several embodiments, the formulations are effective in repairing or otherwise improving the health of a damaged hair cuticle.

In some embodiments, the formulations described herein are used for hair care. In some embodiments, the formulations described herein are used to maintain and/or improve the texture of the hair.

In several embodiments, the hair care formulations described herein can include vasodilators (e.g., Peppermint oil), that can increase blood flow to the hair follicle thereby promote hair growth.

In several embodiments, the hair care formulations described herein can be used to reduce sebum buildup, thereby reducing hair loss. In some embodiments, the hair care formulations can include compositions that reduce sebum buildup on the scalp by inhibiting the production of sebum, regulate sebum production (e.g., Jojoba oil) and/or dissolving excess sebum buildup on the scalp.

In several embodiments, the hair care formulations described herein can be used to reduce the effects of dihydrotestosterone (DHT), which is a key contributor to the onset and progression of androgenic alopecia. In some embodiments, the hair care formulations can include DHT blockers (e.g., Rosemary oil, Tea tree oil, Lavender oil), that reduce DHT buildup on the scalp by inhibiting the production of DHT (conversion of testosterone to dihydrotestosterone). In several embodiments, the formulations are configured to break down or otherwise neutralize, cleanse, eliminate or reduce DHT on the skin or scalp.

In several embodiments, the formulations can be applied on a cleansed or a non-cleansed skin. For example, the formulation for treating dry scalp can be applied to scalp that is wet, dry or damp.

In some embodiments of the invention, the anti-inflammatory agents decrease redness and irritation and fortify resistance to inflammation. The anti-inflammatory agents may have antioxidant properties by reacting with reactive oxygen species. The anti-inflammatory agents may also absorb the energy from UV light and are photo-protective and reduce UV-induced free radical damage to skin.

Although preservatives are provided in certain embodiments, the formulations described herein can be manufactured with reduced or no synthetic preservatives. In some embodiments, the formulations are free of one or more of the following: parabens, phthalates, sulfates, mineral oil, gluten, allergens, and irritants.

The agents, ingredients and compounds described herein may be modified natural substances (e.g., isolates, extracts, purified, processed, chemically modified, etc.) or synthetic substances. Methods of using unique combinations of natural substances are also provided.

In several embodiments, the invention comprises a method of treating skin using any one of the formulations described above or below. The use of any of the formulations described herein for treating skin, preventing skin damage, and/or improving skin appearance is provided in several embodiments. Several embodiments also include instructing the method or use of the formulation (e.g., via instructions for use).

The formulations described herein can provide both short-term and long-term benefits according to several embodiments. Beneficial effects from use of the formulations described herein occur upon use, within hours of use, within 1-2 days, 3-4 days, about 7 days, about 14 days, or within about 3 weeks. In several embodiments, the skin characteristics continue to improve over about 2-4 weeks, about 4-6 weeks, about 6-10 weeks, or about 12-16 weeks of use.

In some embodiments, the formulations described herein can be used in one or more of the following ways: shampoo, serum, oil, leave in lotion/cream, leave in mask, gel mask, peel-off mask and/or sheet mask.

In some embodiments, the formulations described herein can be used as leave in formulations on the skin or hair or can be washed off after application. Further, the formulations can be applied on a cleansed skin or a non-cleansed skin. The time between the application and washing off the formulation can be within an hour of use, within 2 hours, 3 hours, about 5 hours. In several embodiments, the formulations provided for herein are suitable for 1, 2, or 3× daily topical application. In one embodiment, the formulation is left on overnight to provide benefits. In several embodiments, the formulations are able to be used on wet or dry hair or scalp. In several embodiments, the formulations are suitable for application to the scalp or hair and can be left on the scalp or hair until a subsequent shower/bath/wash, etc.

In some embodiments, after the formulation is applied to the skin, the formulation can be removed from the skin with water. In certain embodiments, the formulations can be removed without water, which can be accomplished by massaging the cleanser onto the skin, and, for example, wiping any excess cleanser with a dry or wet cloth, paper towel or facial tissue. In several embodiments, the formulations are meant to be applied to the skin, or hair, but not purposefully removed (e.g., a “leave in” formulation).

According to several embodiments, the formulations described herein can be applied by hand, by sponge, by spraying, by applicator, such as a dropper, by brush, or through use of a composition-impregnated wipe or tissue.

In addition, in some embodiments, at least one of the following ingredients is included: a skin conditioning agent, a solvent, a silicone, an emollient, a preservative, a thickener, an antioxidant, an anti-inflammatory agent, and an excipient. In several embodiments, the formulations are silicone and/or preservative-free. Colorants and fragrances may additionally be included. In some embodiments, the formulation further includes one or more of the following: amino acids, peptides, phospholipids, additional vitamins, growth factors, and additional anti-aging compounds. Surfactants, gelling agents, and pH balancers may also be included.

In some embodiments, the formulation comprises, consists essentially of or consists of several (e.g., 4, 6, 8, 10, 12, 15) or all of the following groups of ingredients, or individual ingredients within a group, listed in Table 5 below:

TABLE 5
Formulation for Treatment of Dry Skin

| | % total formula in some |
|---|---|
| | embodiments as, for example, |
| Ingredients | % m/m, % m/v, % w/w, % w/v, % v/v |
| Water | about 80-98% (e.g., 80, 85, 90, 92, 93, |
| | 94, 95, 95.5, 95.6, 95.7, 96, 97, 98%, or |
| | any amount in between) |
| One or more of Propanediol caprylate, Sarcosine | about 0.01-3% (e.g., 0.01, 0.03, 0.05, |
| | 0.07, 0.09, 0.1, 0.15, 0.2, 0.25, 0.3, 0.5, |
| | 0.75, 1, 1.5, 2, 3%, or any amount in |
| | between) |
| One or more of Glycerin, Bisabolol, C13-C15 | about 0.01-7% (e.g., 0.01, 0.02, 0.05, |
| Alkane | 0.1, 0.2, 0.3, 0.5, 0.6, 0.75, 0.8, 1, 1.5, |
| | 1.75,, 2, 2.5, 3, 4, 5, 6, 7%, or any |
| | amount in between) |
| One or more of Ethylhexyl Olivate, Squalane (or | about 0.001-1% (e.g., 0.0001, 0.0005, |
| optionally squalene) | 0.001, 0.004, 0.005, 0.01, 0.05, |
| | 0.1, 0.15, 0.175, 0.2, 0.25, 0.3, 0.4, |
| | 0.5%, or any amount in between) |
| One or more of Arginine, Sodium phytate, | about 0.0001-2% (e.g., 0.0001, 0.0005, |
| Tocopherol | 0.001, 0.004, 0.005, 0.01, 0.05, |
| | 0.1, 0.15, 0.2, 0.25, 0.3, 0.4, 0.5, 1, 1.5, |
| | 2%, or any amount in between) |
| One or more of Acrylates and/or C10-30 alkyl | about 0.01-3% (e.g., 0.01, 0.05, 0.075, |
| acrylate crosspolymer | 0.1, 0.15, 0.2, 0.25, 0.3, 0.5, 1, 2%, or |
| | any amount in between) |
| One or more of Phenoxyethanol, Ethylhexylglycerin | about 0.05-3% (e.g., about 0.05, 0.06, |
| | 0.067, 0.08, 0.09, 0.1, 0.2, 0.3, 0.4, 0.5, |
| | 1, 1.5, 2, 3%, or any amount in |
| | between) |

In some embodiments, the formulation comprises, consists essentially of or consists of several (e.g., 1, 2, 3) or all of the following groups of ingredients, or individual ingredients within a group, listed in Table 6 below:

TABLE 6
Skin and/or Hair Serum

| | % total formula in some |
|---|---|
| | embodiments as, for example, % m/m, |
| Ingredients | % m/v, % w/w, % w/v, % v/v |
| One or more of Ethylhexyl olivate, Squalane (or | about 1-70% (e.g., 1, 1.5, 2, 2.5, 3, 3.5, |
| optionally squalene) | 5, 10, 20, 30, 40, 45, 50, 55, 58, 60, 65, |
| | 70%, or any amount in between |
| C13-15 Alkane | about 30-60% (e.g., 30, 35, 37.5, 38, 39, |
| | 40, 42, 44, 45, 50, 55, 60%, or any |
| | amount in between) |

In several embodiments, the formulations according to each of Tables 5 and 6 comprise a combination of various combination groups and individual ingredients.

In some embodiments, formulations according to each of Tables 5-6 have a pH at 25 degrees Celsius of about 5.5-7.7 (e.g., 6-7).

In the tables above, the ranges provided in the parentheticals include the overlapping ranges between the numbers. For example, a range of 10-50% (e.g., about 15, 25, 30, 35, 40, 45, 50%) includes, for example, ranges between 15-25%, 15-35%, 30-45%, 40-50%, etc. In one embodiment, the percentages provided are % m/m or % w/w.

In one embodiment, the formulation is configured for treatment of dry scalp, for example a hydrating and/or a moisturizing formulation for the scalp. In one embodiment, the formulation is configured for treatment of oily scalp, for example by reducing, preventing or controlling oil buildup in the scalp. In other embodiments, the formulation is configured as a serum, cream, oil, gel, mask, or cream for the scalp skin. In other embodiments, the formulation is configured for treatment of non-scalp skin (e.g., face, body).

In one embodiment, the formulation comprises or consists essentially of water, sodium phytate, acrylates or C10-30 alkyl acrylate crosspolymer, propanediol caprylate, ethylhexyl olivate, squalane, arginine and one or more ingredients selected from the group of bisabolol, phenoxyethanol, ethylhexylglycerin, C13-C15 alkane, sarcosine and glycerin.

In one embodiment, the formulation comprises or consists essentially of a solvent, a grain derived stabilizer, suspending agents and/or thickener, a synthetic copolymer, a palm derived anti-dandruff agent, an emollient derived from olives, an amino acid and one or more ingredients selected from the group of squalane, bisabolol, phenoxyethanol, ethylhexylglycerin, C13-C15 alkane, sarcosine and glycerin.

In several embodiments, the formulation comprises a grain derived stabilizer. In several embodiments, the formulation comprises fulvic acid. In several embodiments, the formulation comprises glycine. In several embodiments, the formulation comprises kelp or kelp derived chelator. In several embodiments, the formulation comprises Ethylenediaminetetraacetic acid (EDTA). In several embodiments, the formulation comprises sodium gluconate. In several embodiments, the combinations of such stabilizers are used. In several embodiments, the formulation comprises a suspending agent and/or thickener. In several embodiments, the formulation comprises a absorptive thickener. In several embodiments, the formulation comprises a swelling thickener. In several embodiments, the formulation comprises a soluble thickener. In several embodiments, the formulation comprises an electrostatic repulsion-based thickener. In several embodiments, the formulation comprises one or more of xantham gum, carbomers or acrylate copolymers. In several embodiments, combinations of such thickeners are used. In several embodiments, the formulation comprises a synthetic polymer. In several embodiments, the formulation comprises a semi-synthetic polymer or copolymer. In several embodiments, the formulation comprises polyglutamic acid. In several embodiments, the formulation comprises a combination of acrylic acid and methacryluic acid monomers. In several embodiments, the formulation comprises acrylamide monomers. In several embodiments, the formulation comprises one or more of xantham gums, celluloses starches, other gums, or other polysaccharides or complex carbohydrates. In several embodiments, combinations of such polymers are used. In some embodiments, the formulation comprises a palm derived anti-dandruff agent. In several embodiments, the formulation comprises an animal derived anti-dandruff agent. In some embodiments, the formulation comprises zinc pyrithione, climbazolea or piroctone olamine. In some embodiments, the formulation comprises caprylic acid. In several embodiments, the formulation comprises an emollient derived from olives. In several embodiments, the formulation comprises a plant and/or fruit derived emollient. In several embodiments, the formulation comprises a nut derived emollient. In several embodiments, the formulation comprises an animal derived emollient. In several embodiments, the formulation comprises one or more of shea butter, argan oil, jojoba oil, cocoa butter, lanolin, soline, chia seed oil or tamanu oil. In several embodiments, the formulation comprises one or more of cococaprylate caprate, butylene glycol dicaprylate dicaprate. In several embodiments, the formulation comprises an amino acid. In several embodiments, the formulation comprises a plant and/or fruit derived amino acid. In several embodiments, the formulation comprises a nut derived amino acid. In several embodiments, the formulation comprises an animal derived amino acid. In some embodiments, the formulation comprises L-citrulline. In several embodiments, the formulation comprises one or more antioxidant. In several embodiments, an animal or plant derived antioxidant is used. In several embodiments, the antioxidant is a triterpene. In several embodiments, the formulation comprises a natural antioxidant, such as squalene. In several embodiments, the formulation comprises squalane. In several embodiments, the squalane is from a non-animal source. For example, in several embodiments, the antioxidant is derived from yeast (e.g., Kluyveromyces lactis, Saccharomyces cerevisiae, or Pseudozyma sp., among others). In several embodiments, the antioxidant is derived from one of olive oil, rice bran oil, red peanut oil, peanut oil, white sesame oil, black sesame oil, coriander seed oil, soybean oil, and/or sunflower oil. In several embodiments, the antioxidant can be produced, for example by bacteria, such as E. coli, or PCC6803. In several embodiments, the antioxidant can be produced, for example by marine organisms, including algae. In several embodiments, the antioxidant is derived from Aurantiochytrium species 18w-13a, while in other embodiments, the antioxidant is derived from Chlamydomonas reinhardtii. In several embodiments, the formulation comprises an emollient (e.g., a light, protective emollient). In several embodiments, the emollient is a branched or unbranched hydrocarbon. In several embodiments, the emollient is a branched or unbranched hydrocarbon derived from a coconut or plant sugar. In several embodiments, the emollient is a branched or unbranched hydrocarbon derived from non-GMO, renewable plant source. In several embodiments, hemisqualane is used as the emollient. In several embodiments, cyclic free or synthetic silicones are used, such as, for example, low viscosity dimethicone and a mixture of C13-C14 isoparaffin. Combinations of such antioxidants are used, in several embodiments. In several embodiments, chamomile essential oil or an oil-soluble calendula or chamomile extract is used in combination with, or in place of, bisabolol. In several embodiments, natural bisabolol is used, though in some embodiments, synthetic bisabolol is used (optionally with less then about 3% farnesol content). In several embodiments, sodium benzoate, ethylhexylglycerin, and/or leuconostoc/radish root ferment filtrate are used in combination with, or in addition to, phenoxyethanol. Extracts from green tea or chicory may also be used to generate phenoxyethanol, in several embodiments. In several embodiments, a glycerine-derived preservative is used in embodiments of the formulation. In several embodiments, the formulation comprises one or more inhibitors of 5 alpha-reductase (reducing the conversion of testosterone to dihydrotestosterone). In several embodiments, the formulation comprises one or more sebum modulators (e.g., reducers of sebum production). In several embodiments, the formulation comprises one or more of cocoa butter, shea butter, avocado oil, and jojoba oil in combination with, or in place of, glycerin.

In one embodiment, the formulation comprises or consists essentially of (i) water in the range of about 85-99% of the total formulation, (ii) sodium phytate in the range of 0.01-0.07% of the total formulation, iii) one or both of acrylates and C10-30 alkyl acrylate crosspolymer in the range of about 0.01-0.5% of the total formulation, iv) propanediol caprylate in the range of about 0.01-2% of the total formulation, v) at least two or all of ethylhexyl olivate, squalane, sarcosine, glycerin, arginine, tocopherol in the range of about 1-3% of the total formulation and vi) preservatives, viscosity increasing/decreasing agents, surfactants, emulsifiers, conditioners, fragrances, colorants, and pH adjusters, collectively in a range of about 0.5-3% of the total formulation.

In one embodiment, the formulation comprises or consists essentially of water in the range of 90-98% (e.g., about 92-97%), sodium phytate in the range of 0.001-3% (e.g., about 0.01-0.75%), acrylates and/or C10-30 alkyl acrylate crosspolymer in the range of about 0.01-0.9% (e.g., about 0.05-0.5%), propanediol caprylate in the range of about 0.01-2% (e.g., about 0.1-1.5%), sarcosine in the range of about 0.01-2% (e.g., about 0.03-1%), glycerin in the range of about 0.5-5% (e.g., about 0.75-3%), phenoxyethanol and/or ethylhexylglycerin in a collective or individual range of about 0.01-3% (e.g., 0.05-2%), bisabolol in the range of about 0.001-2% (e.g., about 0.05-1%), C13-C15 alkane in the range of about 0.001-3% (e.g., about 0.05-1.5%), ethylhexyl olivate and/or squalane in a collective or individual range of about 0.001-2% (e.g., about 0.005-1.0%) and arginine and/or additional amino acids in the range of about 0.01-2% (e.g., about 0.05-1.0%).

In some embodiments, the ratio of sodium phytate to acrylates and/or C10-30 alkyl acrylate crosspolymer is 1:1, 1:2, 1:3, 1:4, 1:5, 1:6, 1:10, 1:20, 1:30, 1:50, 1:100, 1:200, 1:250, 1:500, 1:1000, and in ranges in between. In some embodiments, the ratio of sarcosine to propanediol caprylate is 1:1, 1:2, 1:3, 1:4, 1:5, 1:6, 1:7, 1:8, 1:9, 1:10, 1:20, and in ranges in between. In some embodiments, the ratio of bisabolol to C13-C15 alkane is 1:1, 1:2, 1:3, 1:4, 1:5, 1:6, 1:7, 1:8, 1:9, 1:10, 1:20, and in ranges in between.

In one embodiment, the formulation comprises or consists essentially of water in the range of 90-98% (e.g., about 92-97%), sodium phytate in the range of 0.005-1% (e.g., about 0.01-0.75%), acrylates and/or C10-30 alkyl acrylate crosspolymer in the range of about 0.1-0.5% (e.g., about 0.1-0.45%).

In several embodiments, the formulation further comprises propanediol caprylate in the range of about 0.1-2% (e.g., about 0.1-1.5%) and optionally sarcosine in the range of about 0.01-2% (e.g., about 0.03-1%).

Several embodiments of the formulation further comprise glycerin in the range of about 0.5-3% (e.g., about 1-2.5%). Some embodiments also include phenoxyethanol in the range of about 0.1-1.5% (e.g., 0.5-1.2%), optionally in combination with ethylhexylglycerin in the range of about 0.01-2% (e.g., about 0.05-1.5%) and/or bisabolol in the range of about 0.01-1% (e.g., about 0.05-0.75%).

Some embodiments of the formulation further comprise C13-C15 alkane, or other hydrocarbon chain lengths, in the range of about 0.01-2% (e.g., about 0.05-1.2%).

Ethylhexyl olivate is optionally included in some embodiments, for example in the range of about 0.05-2% (e.g., about 0.07-1%). Squalane (or optionally squalene) is included in some embodiments, for example, in the range of about 0.001-2% (e.g., about 0.005-1.0%).

Several embodiments further comprise individual amino acids or optionally an amino acid complex. In several embodiments, the formulation further comprises arginine in the range of about 0.01-2% (e.g., about 0.05-1.0%).

In one embodiment, the formulation comprises or consists essentially of water in the range of 90-98% (e.g., about 92-97%), one or more chelating agents in the range of 0.005-1% (e.g., about 0.01-0.75%), one or more acrylates or acrylates crosspolymers (including those of varying chain lengths) in the range of about 0.1-0.5% (e.g., about 0.1-0.45%), one or more anti-dandruff agents in the range of about 0.1-2% (e.g., about 0.1-1.5%), sarcosine in the range of about 0.01-2% (e.g., about 0.03-1.5%), glycerin in the range of about 0.5-2% (e.g., about 0.1-1.5%), one or more preservatives in the range of about 0.05-2% (e.g., about 0.05-1.2%), one or more anti-oxidants with anti-inflammatory properties in the range of about 0.01-1% (e.g., about 0.05-0.75%), one or more emollients in the range of about 0.01-2% (e.g., about 0.05-1.2%), one or more emollients in the range of about 0.001-2% (e.g., about 0.005-1.0%) and one or more conditioning agents in the range of about 0.01-2% (e.g., about 0.05-1.0%).

In one embodiment, the formulation is configured as a serum, for example a hydrating and/or a moisturizing serum. In other embodiments, the formulation is configured as an anti-aging skin composition. In several embodiments, the formulation is configured as serum for application to hair in order to improve one or more of the strength, fullness, thickness, shine, and/or volume of the hair.

In one embodiment, the formulation comprises or consists essentially of two or more of Ethylhexyl olivate, squalane and/or C13-C15 alkane.

In one embodiment, the formulation comprises or consists essentially of (i) ethylhexyl olivate in the range of about 30-75% of the total formulation, (ii) squalane in the range of about 1-10% of the total formulation and iii) C13-C15 alkane in the range of 20-60% of the total formulation.

In one embodiment, the formulation comprises or consists essentially of one or more emollients in the range of about 30-70% (e.g., about 40-65%), one or more emollients in the range of about 1-10% (e.g., about 1-7%) and one or more emollients such as alkane in the range of about 20-60% (e.g., about 25-45%).

In several embodiments, the formulation comprises a fruit, nut, or vegetable-derived emollient. In several embodiments, the formulation comprises a natural or synthetic ester of fatty acids derived from a fruit, vegetable or nut oil. In several embodiments, the formulation comprises an olive oil derived emollient. In several embodiments, the formulation comprises a palm oil derived emollient. In several embodiments, the formulation comprises hydrogenated Olea Europaea (Olive) oil unsaponifiables. In several embodiments, combinations of such emollients are used. In several embodiments, the formulation comprises one or more antioxidant. In several embodiments, an animal or plant derived antioxidant is used. In several embodiments, the antioxidant is a triterpene. In several embodiments, the formulation comprises a natural antioxidant, such as squalene. In several embodiments, the formulation comprises squalane. In several embodiments, the squalane is from a non-animal source. For example, in several embodiments, the antioxidant is derived from yeast (e.g., Kluyveromyces lactis, Saccharomyces cerevisiae, or Pseudozyma sp., among others). In several embodiments, the antioxidant is derived from one of olive oil, rice bran oil, red peanut oil, peanut oil, white sesame oil, black sesame oil, coriander seed oil, soybean oil, and/or sunflower oil. In several embodiments, the antioxidant can be produced, for example by bacteria, such as E. coli, or PCC6803. In several embodiments, the antioxidant can be produced, for example by marine organisms, including algae. In several embodiments, the antioxidant is derived from Aurantiochytrium species 18w-13a, while in other embodiments, the antioxidant is derived from Chlamydomonas reinhardtii. In several embodiments, the formulation comprises an emollient (e.g., a light, protective emollient). In several embodiments, the emollient is a branched or unbranched hydrocarbon. In several embodiments, the emollient is a branched or unbranched hydrocarbon derived from a coconut or plant sugar. In several embodiments, the emollient is a branched or unbranched hydrocarbon derived from non-GMO, renewable plant source. In several embodiments, hemisqualane is used as the emollient. In several embodiments, cyclic free or synthetic silicones are used, such as, for example, low viscosity dimethicone and a mixture of C13-C14 isoparaffin. Combinations of such antioxidants are used, in several embodiments. In alternative embodiments, squalene is used in addition to or in place of squalane.

The agents, ingredients and compounds described herein may be modified natural substances (e.g., isolates, extracts, purified, processed, chemically modified, etc.) or synthetic substances. To Applicant's knowledge, several combinations of ingredients disclosed herein represent unique enhanced formulations that are not naturally occurring (e.g., not found in nature in such combination). Moreover, in several embodiments, the individual ingredients may be modified to be structurally and/or functionally different than the naturally occurring species, thereby resulting in markedly unique effects.

Although preservatives are provided in certain embodiments, the formulations described herein can be manufactured with reduced or no synthetic preservatives. In some embodiments, the formulations are free of one or more of the following: parabens, phthalates, sulfates, mineral oil, gluten, allergens, and irritants.

In addition, in some embodiments, at least one of the following ingredients is included: a skin conditioning agent, a solvent, a silicone, an emollient, a preservative, a thickener, an antioxidant, an anti-inflammatory agent, and an excipient. Colorants and fragrances may additionally be included. In some embodiments, the formulation further includes one or more of the following: amino acids, peptides, phospholipids, additional vitamins, growth factors, and additional anti-aging compounds. Surfactants, gelling agents, and pH balancers may also be included.

### Skin Care Formulations as Described Generally Herein

Where percentages are provided for agents, ingredients, and compounds, they can be % m/m, % m/w, % w/w, % m/v, % v/v and variations thereof with respect to the formulation as a whole, unless otherwise indicated.

The use of agents, ingredients and compounds may be used interchangeably herein. In several embodiments, reference to the term “based” includes the recited agent, ingredient or compounds. As used herein, the terms composition and formulation can be used interchangeably. Salt forms of the acids identified herein may be used instead of or in addition to the acid.

With respect to the use of plural and/or singular terms herein, those having skill in the art can translate from the plural to the singular and/or from the singular to the plural as is appropriate to the context and/or application.

As will be understood by one skilled in the art, for any and all purposes, such as in terms of providing a written description, all ranges disclosed herein also encompass any and sub-ranges and combinations of sub-ranges thereof. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth. The phrases “and ranges in between” can include ranges that fall in between the numerical values listed. For example, “1, 2, 3, 10, and ranges in between” can include 1-10, 1-3, 2-10, etc.

While various aspects and embodiments have been disclosed herein, other aspects and embodiments will be apparent to those skilled in the art. The various aspects and embodiments disclosed herein are for purposes of illustration and are not intended to be limiting, with the scope and spirit being indicated by, for example, the following claims.

1-106. (canceled)

107. A topical formulation, wherein the formulation comprises:
ethylhexyl olivate, wherein the ethylhexyl olivate is present in an amount ranging between 30% and 70%;
squalane, or squalene, wherein the squalane or squalene is present in an amount ranging between 1% and 15%; and
at least one saturated hydrocarbon with a chain length ranging from 10 to 20 carbons, wherein the saturated hydrocarbon is present in an amount ranging between 30% to 60%.

108. The topical formulation of claim 107, wherein the ethylhexyl olivate is present in an amount ranging between 40 and 65%.

109. The topical formulation of claim 107, wherein the formulation does not comprise squalene and the squalane is present in an amount ranging from 1% and 10%.

110. The topical formulation of claim 107, wherein the saturated hydrocarbon comprises an alkane.

111. The topical formulation of claim 110, wherein the alkane comprises C13-C15 alkane in an amount between 30% to 50%.

112. The topical formulation of claim 107, wherein the saturated hydrocarbon is present in an amount between 30% to 50%.

113. The topical formulation of claim 107, wherein the formulation is fragrance free.

114. The topical formulation of claim 107, wherein the formulation further comprises fragrance.

115. The topical formulation of claim 107, wherein the formulation is a serum.

116. The topical formulation of claim 107, wherein the formulation is configured to be applied to hair.

117. The topical formulation of claim 107, wherein the formulation is safe for application to eczema-prone skin.

118. The topical formulation of claim 107, wherein the formulation is safe for application to sensitive skin.

119. The topical formulation of claim 107, wherein the formulation is safe for application to acne-prone skin.

120. A method of improving or maintaining the health, shine, strength, thickness, fullness, and/or volume of hair, comprising applying the formulation, or instructing the application of a formulation of claim 107.

121. A topical formulation, wherein the formulation comprises:
one or more emollients and
at least one saturated hydrocarbon with a chain length ranging from 10 to 20 carbons.

122. The topical formulation of claim 121, wherein the one or more emollients is present in an amount ranging between 1% and 70%.

123. The topical formulation of claim 121, wherein the saturated hydrocarbon is present in an amount ranging between 30% to 60%.

124. The topical formulation of claim 121, wherein the formulation is fragrance free.

125. A topical formulation, wherein the formulation comprises:
ethylhexyl olivate, wherein the ethylhexyl olivate is present in an amount ranging between 30% and 70%;
and
C13-15 alkane, wherein C13-15 alkane is present in an amount ranging between 30% to 60%.

126. The topical formulation of claim 125, wherein the C13-15 alkane is present in an amount between about 30% to about 50%.

127. The topical formulation of claim 125, wherein the formulation is fragrance free and wherein the formulation is a serum.

128. The topical formulation of claim 125, wherein the formulation further comprises squalane, or squalene, wherein the squalane or the squalene is present in an amount ranging between 1% and 15%.

129. A method of improving or maintaining the health, shine, strength, thickness, fullness, and/or volume of hair, comprising applying the formulation, or instructing the application of a formulation of claim 125.
